(19) United States
(12) Patent Application Publication
(10) Pub. No.: US 2007/0235346 A1
(43) Pub. Date: Oct. 11, 2007

(54) System and methods for providing corrected analyte concentration measurements

(76) Inventors: Natasha D. Popovich, Pompano Beach, FL (US); Stephen G. Davies, Tamarac, FL (US); Greta Wegner, Pompano Beach, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/401,458

(22) Filed: Apr. 11, 2006

(51) Int. Cl. C12Q 1/00; G01N 33/487

(52) U.S. Cl. 205/777.5; 204/403.01

(57) ABSTRACT

Methods and devices for determining the concentration of a constituent in a physiological sample are provided. The physiological sample is introduced into an electrochemical cell having a working and counter electrode. At least one electrochemical signal is measured based on a reaction taking place at the cell. The preliminary concentration of the constituent is then calculated from the electrochemical signal. This preliminary concentration is then multiplied by a hematocrit correction factor to obtain the constituent concentration in the sample, where the hematocrit correction factor is a function of the at least one electrochemical signal. The subject methods and devices are suited for use in the determination of a wide variety of analytes in a wide variety of samples, and are particularly suited for the determination of analytes in whole blood or derivatives thereof, where an analyte of particular interest is glucose.

### FIELD OF THE INVENTION

The present invention relates to the field of diagnostic testing and, more particularly, to diagnostic testing systems for measuring the concentration of a substance in a sample.

### BACKGROUND OF THE INVENTION

The present disclosure relates to a biosensor system for measuring an analyte in a bodily fluid, such as blood, wherein the system comprises a unique process and system for correcting inaccuracies in sample concentration measurements. For example, the present disclosure provides methods of correcting analyte concentration measurements of bodily fluids.

Electrochemical sensors have long been used to detect and/or measure the presence of substances in a fluid sample. In the most basic sense, electrochemical sensors comprise a reagent mixture containing at least an electron transfer agent (also referred to as an “electron mediator”) and an analyte specific bio-catalytic protein (e.g. a particular enzyme), and one or more electrodes. Such sensors rely on electron transfer between the electron mediator and the electrode surfaces and function by measuring electrochemical redox reactions. When used in an electrochemical biosensor system or device, the electron transfer reactions are transformed into an electrical signal that correlates to the concentration of the analyte being measured in the fluid sample.

The use of such electrochemical sensors to detect analytes in bodily fluids, such as blood or blood derived products, tears, urine, and saliva, has become important, and in some cases, vital to maintain the health of certain individuals. In the health care field, people such as diabetics, for example, have a need to monitor a particular constituent within their bodily fluids. A number of systems are available that allow people to test a body fluid, such as, blood, urine, or saliva, to conveniently monitor the level of a particular fluid constituent, such as, for example, cholesterol, proteins, and glucose. Patients suffering from diabetes, a disorder of the pancreas where insufficient insulin production prevents the proper digestion of sugar, have a need to carefully monitor their blood glucose levels on a daily basis. A number of systems that allow people to conveniently monitor their blood glucose levels are available. Such systems typically include a test strip where the user applies a blood sample and a meter that “reads” the test strip to determine the glucose level in the blood sample. For example, testing and controlling blood glucose for people with diabetes can reduce their risk of serious damage to the eyes, nerves, and kidneys.

An exemplary electrochemical biosensor is described in U.S. Pat. No. 6,743,635 ('635 patent) which is incorporated by reference herein in its entirety. The '635 patent describes an electrochemical biosensor used to measure glucose level in a blood sample. The electrochemical biosensor system is comprised of a test strip and a meter. The test strip includes a sample chamber, a working electrode, a counter electrode, and fill-detect electrodes. A reagent layer is disposed in the sample chamber. The reagent layer contains an enzyme specific for glucose, such as, glucose oxidase, and a mediator, such as, potassium ferricyanide or ruthenium hexaamine. When a user applies a blood sample to the sample chamber on the test strip, the reagents react with the glucose in the blood sample and the meter applies a voltage to the electrodes to cause redox reactions. The meter measures the resulting current that flows between the working and counter electrodes and calculates the glucose level based on the current measurements.

In biosensors that measure a particular constituent level in blood, certain components of the blood can undesirably affect the measurements and lead to inaccuracies in the detected signal. This inaccuracy may result in an incorrect reading, leaving the patient unaware of a potentially dangerous blood sugar level, for example. As one example, the particular blood hematocrit level (i.e. the percentage of the amount of blood that is occupied by red blood cells) can erroneously affect a resulting analyte concentration measurement.

It is known that variations in the volume of red blood cells can cause errors in the glucose readings measured with disposable electrochemical test strips. Typically, a negative bias (i.e., lower calculated analyte concentration) is observed at high hematocrits, while a positive bias (i.e., higher calculated analyte concentration) is observed at low hematocrits (a condition representative of an anemic state). At high hematocrits, for example, the red blood cells may (1) impede the reaction of enzymes and electrochemical mediators, (2) reduce the rate of chemistry dissolution since there less plasma volume to solvate the chemical reactants, and (3) slow down diffusion of the mediator. These factors can result in a lower than expected glucose reading as less current is produced during the electrochemical process. Conversely, at low hematocrits there are less red blood cells interfering with the electrochemical reaction than expected and a higher current can be measured. Since the concentration of red blood cells alters the diffusion of dissolved reactants, faradaic current measurements are impacted. In addition, the blood sample resistance is also hematocrit dependent, which can affect charging current measurements.

Several strategies have been used to reduce or avoid hematocrit based variations on blood glucose readings. For example, test strips have been designed incorporating meshes to remove red blood cells from the samples or have included particles in chemistry formulations in order to increase the viscosity of red blood cell and remove the effect of low hematocrits. These methods have the disadvantages of increasing the cost and complexity of test strips and undesirably increase the time required for accurate glucose measurement. In addition, alternating current (AC) impedance methods have also been developed to measure electrochemical signals at frequencies independent of and hematocrit effect. Such methods suffer from the increased cost and complexity of advanced meters required for signal filtering and analysis.

An additional prior hematocrit correction scheme is described in U.S. Pat. No. 6,475,372. In that method, a two potential pulse sequence is employed to estimate an initial glucose concentration and determine a multiplicative hematocrit correction factor. A hematocrit correction factor is a particular numerical value or equation that is used (such as, for example, by taking the product of the initial measurement and the determined hematocrit correction factor) to correct an initial concentration measurement. More specifically, a first pulse of one polarity is applied to the reaction cell with the sample, followed by a second pulse of an opposite polarity to the reaction cell with the sample.

The current responses resulting from both pulses are measured as a function of time, with pulse widths for the first step ranging from about 3 to 20 seconds, and for the second step from 1 to 10 s. The glucose concentration in the sample is then estimated from the measured current-time transients (i.e. the current response). A blood hematocrit correction factor is determined using statistical methods, such as, from the mathematical fit of a three dimensional plot based on data collected at several glucose concentrations and blood hematocrit levels.

The three dimensional plot is created from the following variables: the ratio of the first average current response value to the second average current response value, the estimated glucose concentration, and the ratio of the YSI determined glucose concentration to the estimated glucose concentration minus a background value. The initial estimated glucose concentration is then multiplied by the calculated blood hematocrit correction factor to determine the reported glucose concentration.

Using the process of U.S. Pat. No. 6,475,372, most data points were found to fall within +/−15% of actual glucose concentrations using the hematocrit correction factor equation. Data processing using this technique, however, is still fairly complicated because both a hematocrit correction factor and an estimated glucose concentration must be determined to establish the corrected glucose value. In addition, the time duration of the first step greatly increases the overall test time of the biosensor, which is undesirable from the user's perspective.

Accordingly, novel systems and methods for providing corrected analyte concentration measurements are desired that overcome the drawbacks of current biosensors and improve upon existing electrochemical biosensor technologies so that measurements are more accurate.

### SUMMARY OF THE INVENTION

Embodiments of the present invention are directed to medical devices for immobilization and/or retrieval of objects within anatomical lumens of the body that obviate one or more of the limitations and disadvantages of prior immobilization and retrieval devices.

One embodiment is directed to a biosensor including a sample reception region for receiving a blood sample and a reaction reagent system. The reaction reagent system includes an oxidation-reduction enzyme specific for the constituent; a first electron mediator capable of being reversibly reduced and oxidized such that a first electrochemical signal resulting from the reduction or oxidation is related to the constituent concentration in the blood sample; and a second electron mediator capable of undergoing an electrochemical redox reaction where a second electrochemical signal produced by oxidation or reduction of the second mediator is not directly related to the constituent concentration in the blood sample. The second electrochemical signal changes based on the hematocrit level of the blood sample.

In various embodiments, the biosensor may include one or more of the following additional features: wherein the constituent is glucose; wherein the first mediator is a ruthenium containing material; wherein the ruthenium containing material comprises hexaamine ruthenium (III) trichloride; wherein the second mediator comprises brilliant cresyl blue; wherein the second mediator comprises gentisic acid (2,5-dihydroxybenzoic acid); wherein the second mediator comprises 2,3,4-trihydroxybenzoic acid; wherein the second mediator does not interfere with the first electrochemical signal; wherein the second mediator is oxidized or reduced in a potential range distinguishable from that of the first mediator; wherein the second electron mediator is oxidized or reduced at a potential having a magnitude at least 0.2 volts greater or less than that used to oxidize or reduce the first electron mediator; wherein the first and second electrochemical signals are electric current signals obtained through multi-step chronoamperometry; wherein the first and second electrochemical signals are electric current signals obtained through square wave voltammetry; wherein the first and second electrochemical signals are electric current signals obtained through differential pulse amperometry; and wherein the first and second electrochemical signals are electric current signals obtained through cyclic voltammetry.

Another embodiment of the invention is directed to a method for determining a constituent concentration in blood including introducing the blood sample into an electrochemical cell. The electrochemical cell may comprise spaced apart working and counter electrodes and a redox reagent system comprising an enzyme. The cell also includes a first electron mediator capable of being reversibly reduced and oxidized such that a first electrochemical signal resulting from the reduction or oxidation is related to the constituent concentration in the blood sample. The cell also includes a second electron mediator capable of capable of undergoing an electrochemical redox reaction where a second electrochemical signal produced by oxidation or reduction of the second mediator is not directly related to the constituent concentration in the blood sample and changes based on the hematocrit level of the blood sample. The method further includes obtaining the first electrochemical signal; obtaining the second electrochemical signal; determining an initial value corresponding to the constituent concentration of the sample using data from the first electrochemical signal; and correcting the initial value corresponding to the constituent concentration of the sample to remove an effect of the hematocrit level of the sample using a statistical correlation algorithm and data from the second electrochemical signal.

In various embodiments, the method may include one or more of the following additional features: wherein the constituent is glucose; wherein correcting the initial value comprises deriving a preliminary constituent concentration from the first and second signals and multiplying the preliminary constituent concentration by a correction factor based on the second electrochemical signal to derive the constituent concentration in the sample, corrected to offset an effect of the hematocrit level of the blood sample; wherein the statistical correlation comprises determining a slope of the second electrochemical signal; wherein the statistical correlation comprises determining a slope of both the first and second electrochemical signals; wherein the first electrochemical signal is obtained by applying to the electrochemical cell, a first electric potential of a magnitude capable of oxidizing or reducing the first electron mediator and not capable of oxidizing or reducing the second electron mediator; wherein the second electrochemical signal is obtained by applying to the electrochemical cell, a second electric potential of a magnitude capable of oxidizing or reducing the second electron mediator and not capable of oxidizing or reducing the first electron mediator; wherein the second electron mediator is oxidized or reduced at a potential having a magnitude at least 0.2 volts greater or less than that used to oxidize or reduce the first electron mediator; wherein obtaining the first and second electrochemical signals comprises using multi-step chronoamperometry; wherein obtaining the first and second electrochemical signals comprises using square wave voltammetry; wherein obtaining the first and second electrochemical signals comprises using differential pulse amperometry; wherein obtaining the first and second electrochemical signals comprises using cyclic voltammetry; wherein the second electron mediator comprises brilliant cresyl blue; wherein the second electron mediator comprises gentisic acid (2,5-dihydroxybenzoic acid); and wherein the second electron mediator comprises 2,3,4-trihydroxybenzoic acid.

Another embodiment of the invention is directed to a method for determining the hematocrit corrected concentration of an analyte in a physiological sample comprising introducing the physiological sample into an electrochemical cell. The electrochemical cell may comprise spaced apart working and counter electrodes and a redox reagent system comprising an enzyme and a mediator. The method also includes applying a first electric potential to the reaction cell and measuring cell current during a first 50 milliseconds of the first electric potential as a function of time to obtain a first time-current transient; applying a second electric potential to said cell, and measuring cell current as a function of time to obtain a second time-current transient; deriving a preliminary analyte concentration from said first and second time-current transients; and multiplying the preliminary analyte concentration by a hematocrit correction factor based on the first and second time-current transient to derive the hematocrit corrected analyte concentration in said sample whereby the hematocrit corrected concentration of said analyte in said sample is determined.

In various embodiments, the method may include one or more of the following additional features: wherein the first electric potential is a negative electric pulse and the second electrical potential is a positive electrical pulse; wherein the first electric potential is an applied pulse having a duration of about 1-10 milliseconds; wherein the preliminary analyte concentration is determined in part based on a current time transient value as sampled at an end of the applied pulse of the first electric potential; wherein the second electric potential is an applied pulse or about 1-4 seconds; and wherein the preliminary analyte concentration is determined in part based on a current time transient value as sampled at an end of the applied pulse of the second electric potential.

Another embodiment of the invention is directed to a method for manufacturing a plurality of test strips, comprising forming a web containing a conductive layer and a base layer and partially forming the plurality of test strips by electrically isolating a first group of conductive components in the conductive layer using a first process. The method further includes subsequently forming the plurality of test strips by electrically isolating a second group of conductive components in the conductive layer using a second process wherein first and second processes are not the same. The method also includes forming a reagent layer including an enzyme; a first electron mediator capable of being reversibly reduced and oxidized such that a first electrochemical signal resulting from the reduction or oxidation is related to the constituent concentration in the blood sample; and a second electron mediator capable of undergoing an electrochemical redox reaction where a second electrochemical signal produced by oxidation or reduction of the second mediator is not directly related to the constituent concentration in the blood sample and changes based on the hematocrit level of the blood sample.

In various embodiments, the method may include one or more of the following additional features: wherein the web includes a plurality of registration points; wherein the first process includes a laser ablation process; wherein the second process includes a separation process; wherein the separation process includes stamping; wherein the separation process includes separating a plurality of test strips from the web; wherein the plurality of registration points are separated by approximately 9 mm; wherein the plurality of registration points are separated by less than approximately 9 mm; and wherein the first group of conductive components are separated by less than approximately 9 mm.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 is a cyclic voltammogram associated with the use of a gold electrode with a ruthenium hexaamine electron mediator.

FIG. 2 is a graph depicting the change in current response over time during the application of an input voltage pulse in a sample measurement.

FIG. 3A provides a quadratic fit surface plot from measured data and YSI measured concentration values derived from samples at multiple analyte concentrations and blood hematocrit levels.

FIG. 3B provides a graph depicting the percent bias of calculated glucose concentration values compared with YSI measured sample concentration values at various hematocrit levels and analyte concentration levels.

FIG. 3C provides a best fit surface plot from measured data and % deviation values from YSI glucose concentration values derived from samples at multiple analyte concentrations and blood hematocrit levels.

FIG. 3D is a graph depicting the percent bias of corrected glucose values from YSI measured sample concentration values at various hematocrit levels and analyte concentration levels, according to one embodiment of the invention.

FIG. 4A provides a Taylor series fit surface plot from measured data and YSI measured concentration values derived from samples at multiple analyte concentrations and blood hematocrit levels in a single pulse method.

FIG. 4B is a graph depicting the percent bias of calculated glucose concentration values from YSI measured sample concentration values at various hematocrit levels and analyte concentration levels in a single pulse method.

FIG. 5 is a graph depicting the relationship between a particular amperometricly derived ratio and the particular blood sample hematocrit level at various analyte concentration levels.

FIG. 6 is a graph depicting the relationship between a particular amperometricly derived ratio and the YSI measured sample concentration values.

FIG. 7 is a cyclic voltammogram associated with an SRP electron mediator, according to an embodiment of the present disclosure.

FIG. 8A is a linear sweep voltammogram associated with another SRP electron mediator, according to an embodiment of the present disclosure.

FIG. 8B depicts two linear sweep voltammograms, comparing the SRP electron mediator of FIG. 8A with another SRP electron mediator, according to an embodiment of the present disclosure.

FIG. 8C is a table depicting corrected measurement values using one particular SRP substance.

FIG. 9 depicts a particular potential input waveform applied at the working electrode relative to a counter electrode, according to an embodiment of the present disclosure.

FIG. 10 is a graph depicting the change in current response over time during the application of the input waveform of FIG. 9 in a sample measurement using a primary redox probe and a secondary redox probe (“SRP”).

FIG. 11 is another graph depicting the change in current response over time during the application of the waveform described in FIG. 9.

FIG. 12 is a table depicting corrected measurement values using a first correction algorithm.

FIG. 13 is a table depicting corrected measurement values using a second correction algorithm.

FIG. 14 is a graph depicting the dependence of an SRP mediator on the particular hematocrit level of blood.

FIG. 15 depicts the relationship between the measured analyte signal magnitude and the actual sample analyte concentration at multiple concentrations of the SRP.

FIG. 16 is a graph showing the derived relationship between a calculated SRP factor and the hematocrit of the sample.

FIG. 17 is a top plan view of a test strip according to an illustrative embodiment of the invention.

FIG. 18 is a cross-sectional view of the test strip of FIG. 17, taken along line 2-2.

FIG. 19 is a top view of a reel or web according to a further illustrative embodiment of the invention.

FIG. 20 is a top view of a card formed from a portion of the reel or web according to a further illustrative embodiment of the invention.

FIG. 21 is a top view of a conductive layer according to an illustrative embodiment of the invention.

FIG. 22 is a top view of a dielectric layer according to an illustrative embodiment of the invention.

FIG. 23 is a diagram of the manufacturing process according to a further illustrative embodiment of the invention.

### DESCRIPTION OF THE EMBODIMENTS

In accordance with the present disclosure provided herein are electrochemical biosensors developed for measuring an analyte in a non-homogenous fluid sample, such as in a food product or in a bodily fluid chosen from blood, urine, saliva and tears. At a minimum, the biosensor includes at least one or more electrodes and a reaction reagent system comprising an electron mediator and an oxidation-reduction enzyme specific for the analyte to be measured. In one embodiment, the electron mediator comprises a ruthenium containing material, such as ruthenium hexaamine (III) trichloride.

As used herein, the phrase “working electrode” is an electrode at which the electrochemical oxidation and/or reduction reaction occurs, e.g., where the analyte, typically the electron mediator, is oxidized or reduced.

“Counter electrode” is an electrode paired with the working electrode. A current of equal magnitude and of opposite polarity to the working electrode passes through the counter electrode.

“YSI” or “YSI values” means a particular analyte concentration as determined using a Yellow Springs Instrument glucose analyzer, such as, for example, the YSI model 2300 Stat Plus.

As noted above, the '635 patent describes an exemplary electrochemical biosensor used to measure glucose level in a blood sample. The electrochemical biosensor system is comprised of a test strip and a meter. The test strip includes a sample chamber, a working electrode, a counter electrode, and fill-detect electrodes. A reagent layer is disposed in the sample chamber, and generally covers at least part of the working electrode as well as the counter electrode. The reagent layer contains an enzyme specific for glucose, such as, glucose oxidase or glucose dehydrogenase, and a mediator, such as, potassium ferricyanide or ruthenium hexamine.

In one example, glucose oxidase is used in the reagent layer. The recitation of glucose oxidase is intended as an example only and other materials can be used without departing from the scope of the invention. For example, glucose dehydrogenase is another enzyme that is used in glucose biosensors. Similarly, while potassium ferricyanide is listed as a possible mediator, other possible mediators are contemplated. For example, additional mediators include, but are not limited to, ruthenium, osmium, and organic redox compounds. In one embodiment, during a sample test, the glucose oxidase initiates a reaction that oxidizes the glucose to gluconic acid and reduces the ferricyanide to ferrocyanide. When an appropriate voltage is applied to a working electrode, relative to a counter electrode, the ferrocyanide is oxidized to ferricyanide, thereby generating a current that is related to the glucose concentration in the blood sample. The meter then calculates the glucose level based on the measured current and displays the calculated glucose level to the user.

Commonly owned co-pending U.S. patent application Ser. No. 11/242,925 (which is incorporated herein by reference in its entirety) discloses the use of ruthenium hexaamine as another potential mediator. When ruthenium hexaamine [Ru(NH₃)₆]³⁺ is used, the glucose oxidase initiates a reaction that oxidizes the glucose to gluconic acid and reduces [Ru(NH₃)₆]³⁺ to [Ru(NH₃)₆]²⁺. In the case of glucose dehydrogenase, the enzyme oxidizes glucose to glucono-1,5-lactone, and reduces [Ru(NH₃)₆]³⁺ to [Ru(NH₃)₆]²⁺. When an appropriate voltage is applied to the working electrode, relative to the counter electrode, the electron mediator is oxidized. For example, ruthenium hexaamine [Ru(NH₃)₆]²⁺ is oxidized to [Ru(NH₃)₆]³⁺, thereby generating a current that is related to the glucose concentration in the blood sample.

The systems and methods of the present application rely on electron transfer between the electron mediator and the electrode surfaces and function by measuring electrochemical redox reactions. As noted above, these electron transfer reactions (such as the ferrocyanide or ruthenium hexaamine reactions described above) are transformed into an electrical signal that correlates to the concentration of the analyte being measured in the fluid sample. More particularly, the electrical signal results from the application of particular electrode potential input (comprised of a single constant pulse or distinct separate pulses at more than one potential) at the working electrode relative to a counter electrode.

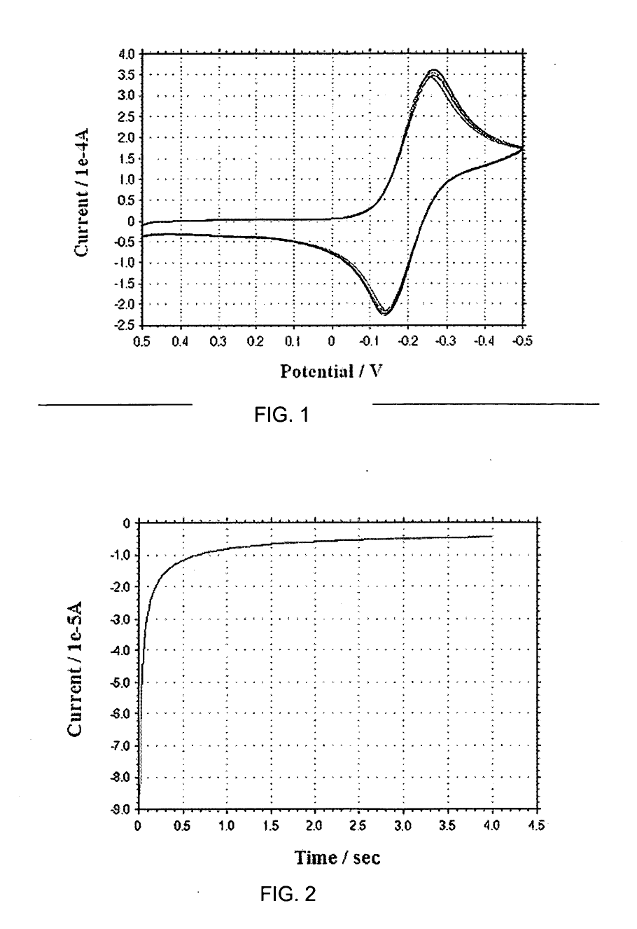

The pulse or pulses are applied to the cell at a particular predetermined potential relative to the redox potential of the particular strip mediator used. As is known in the art, the redox potential of a substance is a measure (in volts) of the substances affinity for electrons (i.e. the substances electronegativity) compared with hydrogen, which is set at zero. Substances capable of oxidizing hydrogen have positive redox potentials. Substances capable of reducing hydrogen have negative redox potentials. One way to determine the particular redox potential of a substance is by cyclic voltammetry. FIG. 1, for example is a cyclic voltammogram associated with the use of a gold electrode with a ruthenium hexaamine electron mediator. As seen in FIG. 1, the ruthenium hexaamine substance exhibits a redox potential of about −0.2 volts vs Ag/AgCl reference electrode in pH 7.25 phosophate buffer solution.

Accordingly, where the desired electron transfer reaction is a reduction of the mediator, for example, a voltage pulse well negative of the redox potential is applied. Conversely, where the desired electron transfer reaction is an oxidation of the mediator, a voltage pulse well positive of the redox potential is applied. The particular electrode potential input into the cell results in an electric signal in the form of a current-time transient. In other words, the final concentration measurement is based on the particular current-time transient (also known as the amperometric current response) obtained as a result of applying a particular voltage potential to the cell (i.e. between the working and counter electrodes) and observing the change in current over time between the working and counter electrodes. FIG. 2, for example, is a graph depicting the change in current response over time during the application of an input voltage pulse in a sample measurement.

The electrochemical method described above with regard to U.S. Pat. No. 6,475,372 is inherently based on a correction for the contribution of hematocrit on the faradaic current generated by the negative input pulse (reduction of the mediator) or the positive input pulse (oxidation of the mediator that was reduced as part of the enzyme-glucose reaction). Initially, the current is composed of contributions from both the charging of the electrical layers of the cell and the diffusion limited (faradaic) current. By the time the current of the first pulse is sampled at time T=100 ms, the charging current has decayed and only the faradaic current remains. The faradaic current can be generally described by the Cottrell equation, Equation No. 1:
i(t)=(nFAD^{1/2}C₀)/(π^{1/2}t^{1/2})
where n is the number of transferred electrons, F is Faraday's constant, A is the electrode area, D is the diffusion coefficient, and C₀ is the initial analyte concentration. Since the effective diffusion coefficient of the analyte is dependent on hematocrit, the measured faradaic current responses of pulses 1 and 2 are used in the method of U.S. Pat. No. 6,475,372 to model hematocrit dependence.

Faradaic vs. Charging Current Hematocrit Correction

The following aspect of the present invention provides an electrochemical method to measure glucose with reduced hematocrit effect. In one embodiment of this method, a negative potential with a pulse width of a few milliseconds (such as, for example, 1-10 ms, but including pulses of duration up to approximately 40 ms) is applied to the electrochemical cell, followed by a positive potential having a duration of about 4 seconds (but including pulses of duration up to 10 seconds). With regard to exemplary potential magnitudes, for ruthenium hexamine, a negative pulse ranging from approximately −0.2 to −0.45 V may be employed with a preferred potential of approximately −0.3 or −0.35 V. A second positive pulse may range from 0.2 to 0.4 V with a preferred potential of approximately 0.3 V. Naturally, the optimal range is directly related to the mediator. For example, if alternate mediators are utilized the optimal positive and negative potential pulses will be related to the oxidation and reduction properties of this mediator.

The current is sampled near the end of both pulse widths. At the end of the first pulse, the charging current is a significant component of the measured current. In general, electrochemical pulse methods have shown that charging current exponentially decays to zero for most systems within 40 ms, while the faradaic current decays much more slowly. The charging current can be described by the following equation, Equation No. 2:
i=E/Rₛ*e[−t/(Rₛ*C_{d})]

where E is the applied potential, Rₛ is the solution resistance, and C_{d} is the electrode layer capacitance. Using this equation, both the solution resistance variable and perhaps the capacitance is dependent on hematocrit and can therefore be manipulated via statistical analysis to determine a hematocrit correction factor.

Accordingly, in the current method, the first pulse results in a current response primarily described by Equation No. 2, while the second pulse results in a current response primarily described by Equation No. 1, described above. By analyzing the first current response based on Equation No. 2, the hematocrit dependent variables of solution resistance and capacitance can be analyzed via statistical analysis (e.g. with a best fit correlation, such as a Taylor Series fit) to help determine a hematocrit correction factor. In addition, using the second current response and Equation No. 1, the hematocrit dependent variable of diffusion can also be analyzed via statistical analysis to assist in determining a hematocrit correction factor.

Accordingly, in one embodiment of the current invention, a pulse 1 current response, P1, (recorded, for example, at t=5 ms) and a pulse 2 current response, P2, (recorded, for example, at t=4 s) are measured resulting from tests performed on multiple fluid samples. These initial measurements may be performed using a particular lot of test strips. Then multiple samples, having known glucose concentration levels, are tested to determine and record the P1 and P2 current values for multiple glucose concentration values. These known glucose concentration levels of the samples are then correlated with particular variables based on the P1 and P2 data.

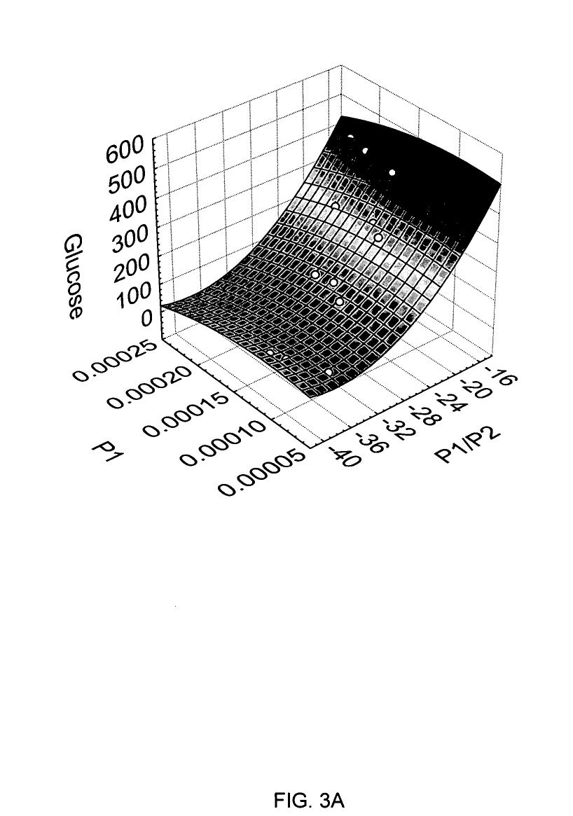

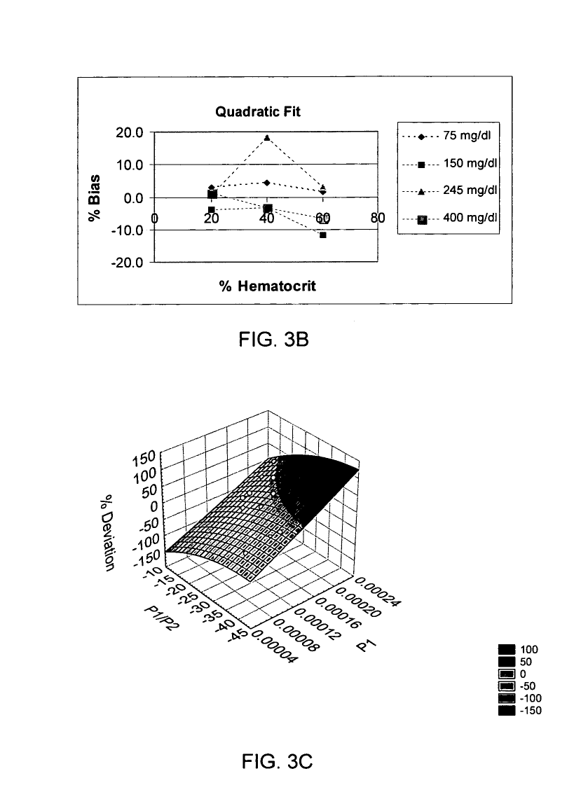

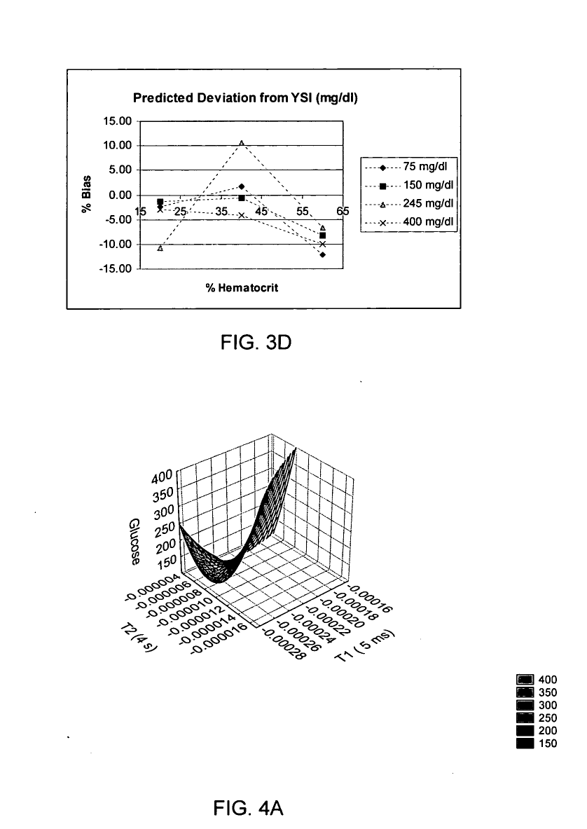

For example, FIG. 3A depicts a three-dimensional quadratic surface plot fit based on a correlation of P1 and P2 data collected at several glucose concentrations and blood hematocrit levels. In FIG. 3A, the variable of the P1 and P2 current ratio (P1/P2 as depicted along the X axis) and the variable of the P1 current (P1 as depicted along the Y axis) are correlated (with a quadratic least squares best fit, for example) with the known sample glucose concentration levels (the (mg/dl) concentration values along the Z axis) resulting in the surface plot displayed. Thereafter, for all strips in the given lot, the calculating meter is programmed with the corresponding surface plot fit. When used to measure P1 charging current and P2 faradaic current of a particular sample, the meter calculates the appropriate glucose level according to the surface plot correlation, which is then displayed on the meter. Alternate mathematical interpretations may be employed. For example, the relationship between P1 currents, P2 currents, and YSI or between P1/P2, P1, YSI may be correlated.

In a test experiment, test glucose concentration values for particular fluid samples were determined by inputting P1 and P1/P2 values into the quadratic fit equation. The resulting test data was compared with the actual YSI measured glucose concentration data for the fluid samples. As seen in FIG. 3B, the samples tested included fluids having varying glucose concentrations and varying hematocrit levels. The test values obtained were within +/−20% of the YSI measured glucose values, as depicted in FIG. 3B.

Turning to FIG. 3C, one system of correcting for the hematocrit effect on analyte measurements is provided. The measured current is dependent on the % hematocrit in a given blood sample with higher currents observed for low hematocrits, and lower current observed at high hematocrits. Variations between the analyte concentrations calculated in the procedure above and the actual YSI glucose values can be mathematically related to the P1 (5 ms)/P2 (4 s) values and the P1 (5 ms) values in a three-dimensional plot. Other mathematical configurations that relate the charging and faradaic currents to YSI deviations may prove to be preferable to using alternate sensor designs (e.g. employing the ratio of pulse 1 and pulse 2, as described with reference to FIG. 5 below).

In FIG. 3C, the variable of the P1 current (P1 as depicted along the X axis) and the variable of the P1 and P2 current ratio (P1/P2 as depicted along the Y axis) are correlated (with a quadratic least squares best fit, for example) with the known value of the % deviation between the initially calculated glucose concentration value and the YSI measured glucose concentration levels (along the Z axis) resulting in the surface plot displayed. Using this surface plot, a particular % deviation is determined for each sample. These best fit surface plot correlations can then be used to correct the glucose measured concentration to reduce the offsetting effect of the particular blood hematocrit level.

In the approach using the plot of FIG. 3C, an estimated glucose concentration would be determined from the faradaic current measured at P2 (4 s). Next, the predicted percent deviation from YSI values would be calculated from the P1 (5 ms) current value to assess the extent of hematocrit dependent effects. This value is then used to correct the estimated glucose concentration. The resulting corrected values are depicted in FIG. 3D. More particularly, FIG. 3D provides the results of a comparison between the corrected glucose concentration data and YSI measured glucose concentration values. As seen in FIG. 3D, the bias of the corrected glucose values are depicted for samples at multiple concentration values each at various hematocrit levels. Samples having glucose concentration levels of 75 mg/dl, 150 mg/dl, 245 mg/dl, and 400 mg/dl were tested, each at three different hematocrit percentage levels. The resulting % bias of calculated corrected glucose values deviating from the YSI values are shown in FIG. 3D to be within +15% and −15%.

Table one, directly below, presents raw data from the above described two-pulse methods of determining an analyte concentration using a charging vs. faradaic current measurement technique.

TABLE 1

| | | |
|---|---|---|
| Two Pulse Data | P1 (5 ms) | P2 (4 s) |

| Glucose | HCT | Ave | StDev | % CV | Ave | StDev | % CV |
|---|---|---|---|---|---|---|---|
| 75 | 20 | 1.16E−04 | 1.29E−05 | 11.14 | 2.91E−06 | 1.19E−07 | 4.10 |
| 75 | 40 | 1.03E−04 | 1.06E−05 | 10.24 | 2.63E−06 | 9.08E−08 | 3.45 |
| 75 | 60 | 6.82E−05 | 1.40E−05 | 20.57 | 1.83E−06 | 1.82E−07 | 9.93 |
| 150 | 20 | 1.64E−04 | 8.39E−06 | 5.12 | 5.76E−06 | 2.45E−07 | 4.26 |
| 150 | 40 | 1.44E−04 | 8.34E−06 | 5.78 | 4.99E−06 | 1.72E−07 | 3.45 |
| 150 | 60 | 1.17E−04 | 1.08E−05 | 9.23 | 3.94E−06 | 1.94E−07 | 4.91 |
| 245 | 20 | 1.97E−04 | 1.12E−05 | 5.69 | 9.30E−06 | 3.52E−07 | 3.78 |
| 245 | 40 | 1.62E−04 | 1.18E−05 | 7.30 | 7.67E−06 | 1.91E−07 | 2.49 |
| 245 | 60 | 1.35E−04 | 1.08E−05 | 7.96 | 6.11E−06 | 3.13E−07 | 5.13 |
| 400 | 20 | 2.26E−04 | 1.40E−05 | 6.20 | 1.47E−05 | 5.26E−07 | 3.57 |
| 400 | 40 | 1.94E−04 | 1.88E−05 | 9.69 | 1.21E−05 | 3.49E−07 | 2.88 |
| 400 | 60 | 1.55E−04 | 2.24E−05 | 14.5 | 8.85E−06 | 3.35E−07 | 3.8 |

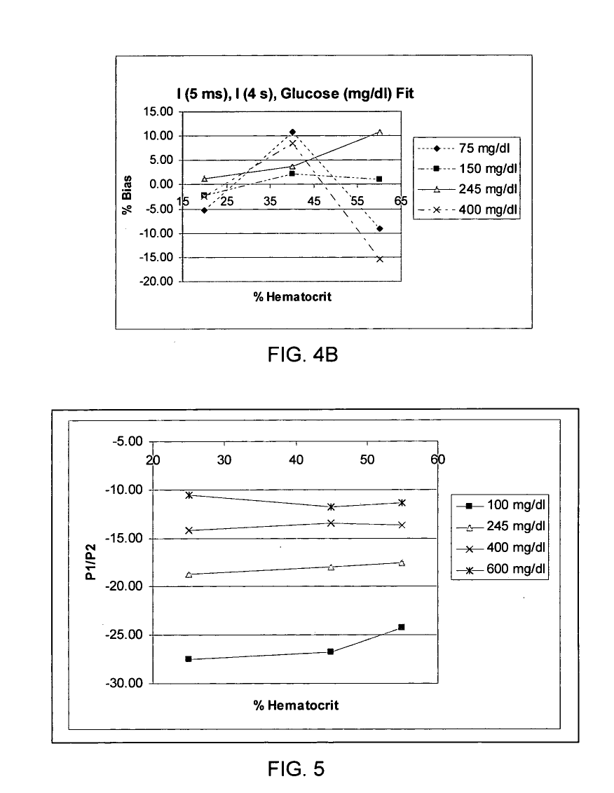

In another variation, instead of applying two distinct pulses, both the charging and faradaic current of a single electrochemical reaction (i.e. the application of a single pulse) may be used to calculate a corrected glucose value. For example, when a 0.3 V potential is applied to a sensor containing ruthenium hexamine, charging current data may be collected at 5 ms I_{(T1)} into the reaction along with faradaic current at 4 s I_{(T2)}. I_{(T1)} and I_{(T2)} may be mathematically related to YSI glucose concentrations using a Taylor Series type of least squares fit. One example of such a Taylor Series fit is depicted in FIG. 4A.

FIG. 4A depicts a three-dimensional Taylor Series fit based on a correlation of I_{(T1)} and I_{(T2)} data collected at several glucose concentrations and blood hematocrit levels. In FIG. 4A, the variable of I_{(T1)} (depicted along the X axis) and the variable of I_{(T2)} (depicted along the Y axis) are correlated (with a Taylor Series least squares best fit, for example) with the known sample glucose concentration levels (the (mg/dl) concentration values along the Z axis) resulting in the surface plot displayed. Thereafter, final glucose concentration values (mg/dl) of the samples were obtained by inputting the I_{(T1)} and I_{(T2)} values into the Taylor Series fit.

The resulting glucose values displayed reduced bias with respect to measured YSI values (mg/dl). FIG. 4B is a graph depicting the percent bias of the calculated glucose concentration values using the single pulse technique described in the preceding two paragraphs. As seen in FIG. 4B, the bias of the corrected glucose values are depicted for samples at multiple concentration values each at various hematocrit levels. Samples having glucose concentration levels of 75 mg/dl, 150 mg/dl, 245 mg/dl, and 400 mg/dl were tested, each at three different hematocrit percentage levels. The resulting % bias of calculated corrected glucose values deviating from the YSI values are shown in FIG. 4B to be within +15% and −15%.

Table two directly below presents raw data from the above described one-pulse method charging vs. faradaic current measurement.

TABLE 2

One Pulse Data

| | | |
|---|---|---|
| YSI | P1 (5 ms) | P2 (4 s) |

| Glucose | HCT | (mg/dl) | Ave | StDev | % CV | Ave | StDev | % CV |
|---|---|---|---|---|---|---|---|---|
| 75 | 20 | 80.9 | −1.55E−04 | 8.34E−06 | 5.38 | −3.70E−06 | 9.15E−08 | 2.47 |
| 75 | 40 | 73.6 | −1.19E−04 | 1.54E−05 | 12.93 | −2.84E−06 | 9.85E−08 | 3.46 |
| 75 | 60 | 78.9 | −1.00E−04 | 8.20E−06 | 8.18 | −2.20E−06 | 1.90E−07 | 8.66 |
| 150 | 20 | 163.3 | −2.14E−04 | 1.61E−05 | 7.53 | −7.08E−06 | 2.89E−07 | 4.09 |
| 150 | 40 | 166.5 | −1.80E−04 | 1.52E−05 | 8.43 | −6.01E−06 | 1.89E−07 | 3.14 |
| 150 | 60 | 165.3 | −1.36E−04 | 9.16E−06 | 6.74 | −4.41E−06 | 2.16E−07 | 4.91 |
| 245 | 20 | 237.9 | −2.47E−04 | 1.57E−05 | 6.36 | −1.08E−05 | 6.70E−07 | 6.20 |
| 245 | 40 | 263.3 | −2.05E−04 | 2.15E−05 | 10.46 | −8.85E−06 | 3.66E−07 | 4.13 |
| 245 | 60 | 260.5 | −1.54E−04 | 1.89E−05 | 12.30 | −6.59E−06 | 2.76E−07 | 4.18 |
| 400 | 20 | 408.1 | −2.85E−04 | 1.89E−05 | 6.64 | −1.71E−05 | 8.01E−07 | 4.68 |
| 400 | 40 | 403.2 | −2.30E−04 | 2.41E−05 | 10.51 | −1.29E−05 | 8.00E−07 | 6.23 |
| 400 | 60 | 420.0 | −1.87E−04 | 1.74E−05 | 9.32 | −9.05E−06 | 4.89E−07 | 5.40 |

In another aspect of this system and method, analyte concentration values may be determined directly from the ratios of pulse 1 current response (taken in this instance at t=2 ms) (P1) and pulse 2 current response (taken in this instance at t=4 s) (P2). With reference to FIG. 5, a graph is provided depicting the resulting ratio of P1/P2 for samples at multiple concentration values, and each at various hematocrit levels. Samples having glucose concentration levels of 100 mg/dl, 245 mg/dl, 400 mg/dl, and 600 mg/di were tested, and each at three different hematocrit percentage levels. As seen in FIG. 5, the resulting ratios were revealed to not be dependent on hematocrit level variations, as evidenced by the relatively constant ratio for each concentration line. Importantly, however, this ratio is revealed to be dependent on the actual glucose concentration of the sample.

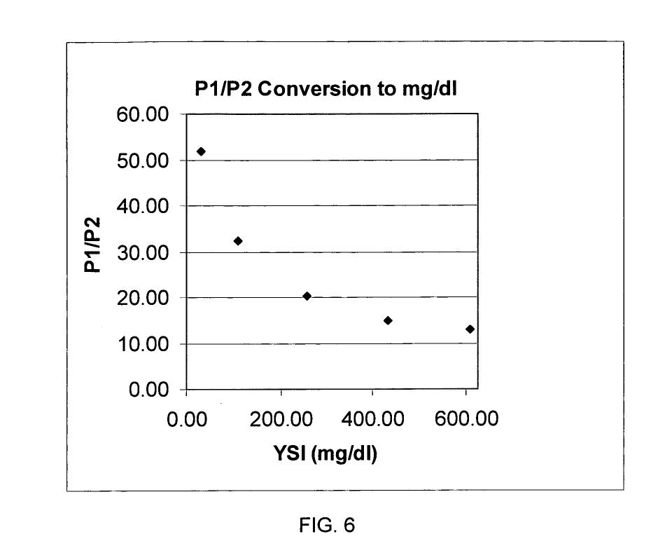

FIG. 6, for example, provides a graph of P1/P2 current ratios versus YSI glucose values in mg/dl. This plot indicates that there is a correlation between experimentally measured P1/P2 current ratios and the actual YSI glucose concentration sample values. Accordingly, statistical methods can be used to mathematically convert the measured ratio to a particular glucose concentration value.

Secondary Redox Probe Hematocrit Correction Approach

As noted earlier, during a sample test, the glucose dehydrogenase initiates a reaction that oxidizes the glucose to glucono-1,5-lactone and reduces [Ru(NH₃)₆]³⁺ to [Ru(NH₃)₆]²⁺. When an appropriate voltage is applied to a working electrode, relative to a counter electrode, the [Ru(NH₃)₆]²⁺ is oxidized to [Ru(NH₃)₆]³⁺, thereby generating a current that is related to the glucose concentration in the blood sample. The current generated is necessarily dependent on the glucose concentration of the sample. Using this relationship, the glucose level can be displayed using a simple correlation algorithm. As also noted above, however, the particular blood hematocrit level can erroneously affect a resulting analyte concentration measurement. Accordingly, an additional method of hematocrit correction has been developed based on the addition of a secondary redox probe (“SRP”) into strip chemistry. For purposes of this disclosure, “redox probe” means a substance capable being oxidized and/or reduced.

In the following disclosure, the measurement technique examined is multi-step chronoamperometry. However, there are other types of measurement that would be amenable to use in the invention. For example square wave voltammetry, differential pulse amperometry, and cyclic voltammetry are all contemplated to be viable means of measurement in the invention. It is not the intention to limit the scope of this invention to a particular measurement method.

The particular secondary redox probe can comprise an additional electron mediator substance capable of undergoing an electrochemical redox reaction. Accordingly, in the same manner as the ruthenium hexaamine mediator mentioned above, the secondary redox probe substance generates a current in response to the application of a voltage pulse. The secondary redox probe, however, differs from the ruthenium hexaamine (i.e. the primary redox probe), or the other mediators cited above, in that the current generated is instead unrelated to the glucose concentration, but still dependent on the particular blood hematocrit level of the sample.

Accordingly, the electrochemical signal produced by the SRP will be a function of the hematocrit of the sample, but not glucose dependant, and it will therefore function as an internal standard for hematocrit evaluation. This information can be used to correct the glucose signal for the hematocrit effect as will be described below.

Some of the classes of compounds that could function as a SRP include transition metal complexes, organometallics, organic dyes and other organic redox-active molecules. The following is an exemplary list of characteristics for the SRP. Although preferred, it is not required that the SRP exhibit all of the following characteristics.
The SRP should not interfere with the glucose measurement (i.e., limited interaction with the enzyme, mediator, or glucose).
The SRP should be oxidized or reduced in a potential range that can be easily distinguished from that of the mediator.
The SRP should be soluble in the strip chemistry formulation.
The SRP should not degrade the stability of the sensor, or any other performance parameter.

For an electrochemically active compound to be useful as an SRP, it should have a potential distinctly different from the primary mediator, but not so extreme that measuring it would result in a noisy signal due to interference. For example, when ruthenium hexaamine is used as the mediator, there are two preferable (but not required) ‘windows’ in the potential range. In an oxidation based approach, one of the windows is from about 0.3 to approximately 0.9V. The second window is the reduction-based technique, and extends from approximately −0.15V to −0.5V. It is important to remember that the numbers cited here are only for a very specific example, and should not be construed as a general rule. There may be cases where an SRP that has a peak at 0.2V, or at other magnitudes, would be perfectly acceptable. The actual range of the windows is dependent on the potential required for the primary measurement.

Beyond the scope of hematocrit dependence, potential ranges, and a preference for avoiding interference with the primary measurement, there are few restrictions on what exactly can be used as an SRP. This enables the use of a wide variety of substances, including, but not limited to: simple organics, macromolecules, functionalized microbeads, transition metal complexes, nanoparticles, and simple ions.

The SRP is used during a sample measurement by applying a two-step potential waveform. In the first step, the signal of the primary probe is measured on the working electrode in the standard manner. After this initial pulse, a second, different potential pulse, is applied to the working electrode. This second pulse is designed to measure the signal of the Secondary Redox Probe (“SRP”). The signal is then processed to give a factor that can be compared to a standard value. This will allow the meter's software to correct the value of the primary measurement. The pulses can be either negative potential (reduction), or positive potential (oxidation). The preferred type of SRP depends on the primary probe used. In the case of the sensors in this specific embodiment, an oxidation-based SRP is advantageous in that an oxidation-based SRP is easier to implement than the reduction SRP because the primary measurement step is the same as the SRP detection step, thus allowing the SRP measurement to occur on the same set of electrodes as that used by the primary measurement.

The use of an oxidation based SRP therefore obviates the need to use the fill detect electrodes to form a four-electrode system which would be required for a reduction-based SRP. This simplifies meter design and provides other advantages as well. For instance, since the electrodes measuring the SRP are the same as those used in the primary measurement, and on the same time scale, in the same sample, it very accurately reflects the conditions experienced by the primary redox probe.

Using a reduction based SRP for an oxidation-based system, however, is certainly possible. Reduction measurements would be conducted on fill detect electrodes by applying a two-step potential waveform. In this example, in the first step, ruthenium(III) that is present in the sample would be reduced to ruthenium(II) so that it does not interfere with the measurement of the SRP. The second step would be to a more negative potential at which the SRP is reduced. This signal would then be measured to determine hematocrit correction. As noted above, the SRP should have a reduction potential that is significantly different from the reduction potential of the primary mediator (i.e. ruthenium(II) for example). The SRP potential should be negative enough to completely reduce the SRP, while not being so negative that it starts to cause large amounts of background noise. Signal measured with the reduction approach can become limited by the amount of Ru(II) that is present at the electrode that serves as the counter electrode, and is glucose dependent at low glucose levels.

In the case of oxidation, the same two-step potential approach could be utilized. In this case, the measurement could be conducted using the primary measurement anode as the working electrode. The first potential step would oxidize ruthenium(II) resulting from the glucose reaction. The potential would then be increased to a higher magnitude required for oxidation of the SRP.

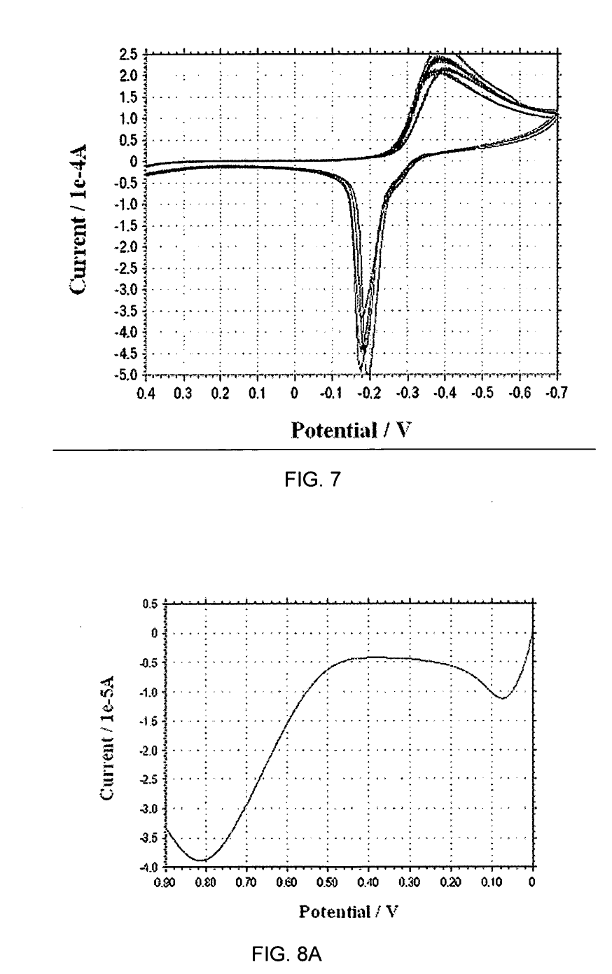

FIG. 7 is a cyclic voltammogram associated with an SRP electron mediator where brilliant cresyl blue, an organic dye, is the selected SRP substance. Comparing FIG. 7 and FIG. 1 demonstrates that brilliant cresyl blue has at least a reduction peak significantly different from that of the ruthenium hexaamine mediator. Therefore, when a ruthenium mediator is used as the primary probe, an SRP of brilliant cresyl blue will be easily distinguishable from the primary probe in a reduction based measurement. Therefore, during a measurement, the ruthenium hexaamine mediator can be reduced after the application of a first potential pulse and the brilliant cresyl blue mediator can be reduced later after the application of a second different potential pulse. Brilliant cresyl blue is in this case used as a reduction based SRP.

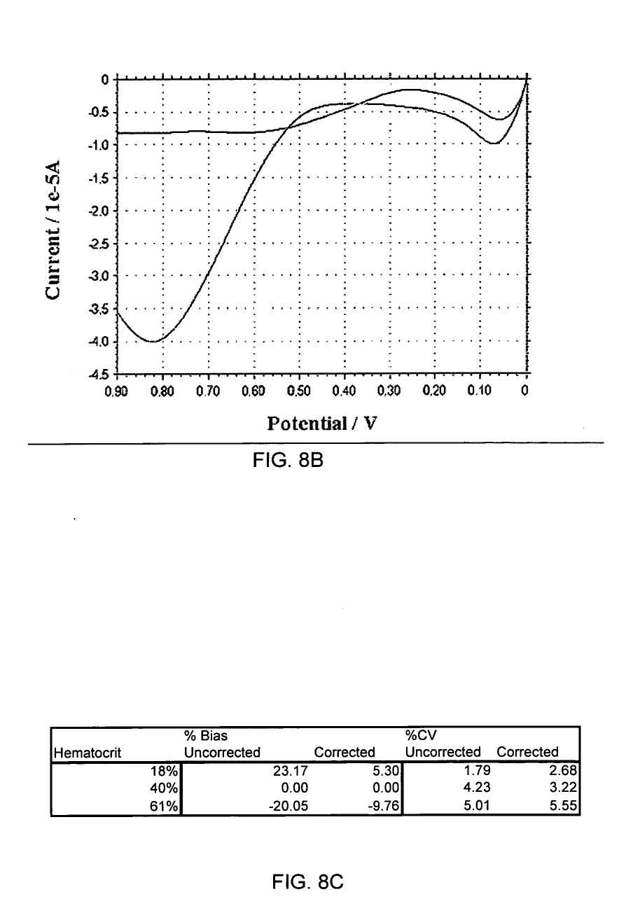

FIG. 8A is a linear sweep voltammogram associated with another potential SRP electron mediator, according to an embodiment of the present disclosure. FIG. 8A depicts a linear sweep voltammogram of the substance gentisic acid (2,5-dihydroxybenzoic acid). Comparing the gentisic acid peak (the leftmost peak), to the Ruthenium peak (the right peak) demonstrates that gentisic acid has at least an oxidation peak (e.g. at approximately 0.81 volts) significantly different from that of the ruthenium hexaamine mediator. Therefore, when a ruthenium mediator is used as the primary probe, an SRP of gentisic acid will be easily distinguishable from the primary probe in an oxidation based measurement. Therefore, during a measurement, the ruthenium hexaamine mediator can be oxidized during the application of a first potential pulse and the gentisic acid mediator can be oxidized later during the application of a second different potential pulse. The foregoing voltammograms successfully demonstrate the use of simple organic compounds as SRPs.

Concentration of the SRP used is dependent on the specific SRP in question. Many times, the concentration is limited by specific attributes of the SRP or the chemistry. For instance, the SRP may only be soluble to a certain concentration, or it may start to affect the primary measurement at higher concentrations. Also important to note is the voltage used to measure the SRP. For voltages with a high magnitude, more background will be produced (due to more interferants being measured), and thus a higher concentration of SRP may be needed to effectively make negligible the background noise contribution. Conversely, an SRP that demonstrates a very intense signal may only require that a small amount be added to observe an adequate signal.

For the purposes of the SRPs mentioned in this embodiment, 5-20 mM of SRP mixed into the chemistry formulation seems to be sufficient to create an adequate signal without unwanted side effects such as alteration of viscosity and consistency of the chemistry solution, or interference with the primary measurement.

Since the SRP method relies on a voltage distinctly different from that of the primary mediator, it may be affected by additional interferants that would not necessarily affect the primary measurement. Interferants will tend to result in an overcorrected hematocrit value (i.e., lower than the actual). This is due to the interferant(s) increasing the apparent concentration of the SRP by contributing to the current measured at the SRP detection step. A higher concentration of an SRP will tend to give a lower response value. This response value is calculated and not a direct measurement.

For one study, two concentrations of the SRP gentisic acid were used in the biosensor chemistry, 10 mM and 20 mM. As evidenced in tables 3 and 4 below, levels for interferants spiked into the blood were at FDA-mandated levels or above. As can be seen in the tables 3 and 4, the 20 mM concentration seems less affected by interferants than the 10 mM. Thus, it may be advantageous to use the highest amount of SRP possible without affecting the primary measurement or going above the saturation point of the chemistry involved.

The Salicylate is easily the interferant with the most impact on the measurement. The other interferants do not register outside the margin of error for the 20 mM. For the 10 mM, the acetaminophen and the ascorbic acid register slightly, but their effect is not as pronounced as that of salicylate. In terms of the effect on the actual correction, the salicylate, being the most noticeable, could generate a measured shift of approximately 10 hematocrit points for the 10 mM gentisic acid formulation, and 4-6 points for the 20 mM gentisic acid formulation.

TABLE 3
20 mM SRP concentration

| Interferant | Response | % Bias from Control |
|---|---|---|
| Control | 0.1394 | 0.0 |
| Acetaminophen | 0.1401 | −0.5 |
| Ascorbic Acid | 0.1393 | 0.1 |
| Salicylate | 0.1347 | 3.4 |
| Uric Acid | 0.1366 | 2.1 |

TABLE 4
10 mM SRP concentration

| Interferant | Response | % Bias from Control |
|---|---|---|
| Control | 0.1989 | 0.0 |
| Acetaminophen | 0.1936 | 2.7 |
| Ascorbic Acid | 0.1935 | 2.7 |
| Salicylate | 0.1867 | 6.2 |
| Uric Acid | 0.1987 | 0.1 |

In the SRP method, the second pulse potential is carefully selected. Biological fluids, such as, for example, blood, are very complex matrices, and many interferants may be present. Interferants may cause a shift in the SRP signal, which would lead to an erroneous correction. An erroneous measurement could result in a health risk for the end user. Therefore, it is advantageous to use an SRP substance with as low a redox potential magnitude as possible for a given measurement. The reasoning for this is that at lower redox potential magnitudes, less of the possible pool of interferants undergo redox reactions. Therefore, the resultant response is less likely to be erroneous due to the effect of unintended redox reactions occurring in the interferants.

At the same time, a basic requirement for an SRP is that it have a potential distinctly different from that of the primary redox probe. Therefore, the lower boundary for the magnitude of a particular SRP candidate's redox potential for any particular system would be the potential at which the primary probe is measured.

For purposes of exposition, two oxidation-based SRP substances are compared in FIG. 8B. In FIG. 8B, one SRP is gentisic acid, disclosed previously in FIG. 8A. The other is 2,3,4-trihydroxybenzoic acid, a derivative of gentisic acid. These two SRPs are structurally similar, but 2,3,4-trihydroxybenzoic acid has a lower redox potential. The linear sweep voltammograms of FIG. 8B show two blood samples, one containing gentisic acid chemistry (the curve exhibiting a Y-axis value of about −3.4 at a potential of 0.9), and one containing 2,3,4-trihydroxybenzoic acid chemistry (the curve exhibiting a Y-axis value of about −0.7 at a potential of 0.9). Differences in magnitude of the peaks should be ignored, as the scan rates is five times as slow for the 2,3,4-trihydroxybenzoic acid sweep, resulting in lower magnitude signal. An examination of FIG. 8B reveals that 2,3,4-trihydroxybenzoic acid has a redox peak near 0.63V, while gentisic acid has a peak near 0.83V. This 0.2V difference in potential can be significant. A chronoamperometric examination of the background signal can reveal the difference in redox peaks.

FIG. 8C shows the results of a trial using samples with 3 separate hematocrits at a 245 mg/dL glucose concentration and 2,3,4-trihydroxybenzoic acid as the SRP detected at 0.65V. The results are comparable to gentisic acid. Therefore, in the absence of evidence of disadvantageous properties, 2,3,4-trihydroxybenzoic acid would be seen as useful alternative to gentisic acid as the SRP substance since 2,3,4-trihydroxybenzoic acid exhibits a lower redox potential magnitude.

As noted above, two approaches can be used for detection of the SRP: reduction and oxidation. In the case of oxidation-based systems, reduction measurements would be conducted on fill detect electrodes by applying a two-step potential waveform. If the system in question were a reduction-based biosensor, a reduction-based SRP would be the more advantageous, and would be conducted on the primary electrodes. In the first step, the primary mediator, in this case ruthenium(III), that is present in the sample would be reduced to ruthenium(II) so that it does not interfere with the measurement of the second redox probe. The second step would be to a more negative potential at which the second redox probe is reduced. This signal would then be measured to determine hematocrit correction. In this case, the SRP should have a reduction potential that is significantly different from the reduction potential of ruthenium(III). This potential should be negative enough to completely reduce the SRP, while not being so negative that it starts to cause large amounts of background noise.

In the case of oxidation, the same two-step potential approach could be utilized. In this case, the measurement could be conducted using the anode as the working electrode. The first potential step would oxidize ruthenium(II) resulting from the glucose reaction. The potential would then be increased to a higher value required for oxidation of the secondary redox probe.

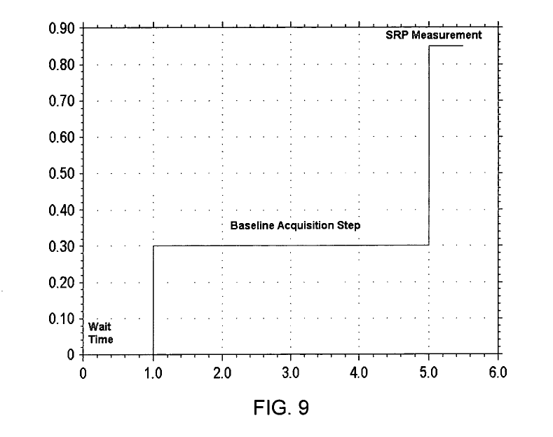

FIG. 9 depicts a particular potential input waveform applied at the working electrode relative to a counter electrode, according to an embodiment of the present disclosure. As seen in FIG. 9, in one embodiment the pulsing method for SRPs consists of three steps. The first step is optional, and is referred to as mixing time, or wait time. Zero potential is applied to the electrodes, and this essentially gives the reaction cell contents time to dissolve and mix evenly. This is not required for the SRP method, but is used in some embodiments in order to provide optimal reaction conditions. The next step is the primary redox probe measurement step, also called the suppression step.

The suppression step establishes a current baseline for the subsequent SRP step. Since in the example cited, the SRP measurement is performed on the same electrode pair as our primary measurement (i.e. in this example both are oxidation based reactions), this step has a dual purpose. It is both a means of establishing an SRP baseline and it is also the primary measurement. As seen in FIG. 9, the first step applies a constant voltage pulse of about 0.30 volts for about 4 seconds. The primary measurement step can last any amount of time, but it is found to be advantageous for it to be at least 3-4 seconds long. Very short detection steps result in increased error in both the primary and SRP measurements.

The final step is the secondary redox probe measurement step. The voltage is changed and the current response generated by the SRP is measured. This, along with the baseline, is entered into an equation and a value that describes the hematocrit level is obtained. As seen in FIG. 9, in one example, the second step applies a constant voltage pulse of about 0.85 volts for a predetermined span of time. The SRP step can be almost any length of time. However, it is most advantageous to keep the test time as short as possible for the consumer, thus a test time of 0.1 s to 1 s would be considered typical for the purposes of the examples cited above. Again, shorter times are possible, and have been shown to measure differences in hematocrit. However, this short measurement may result in increased equipment cost.

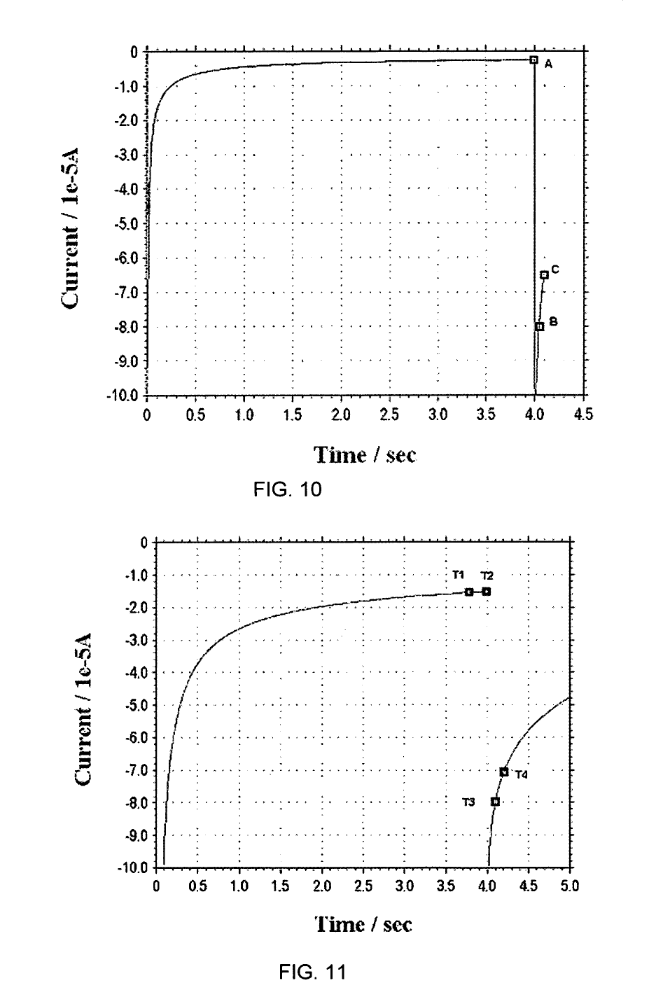

FIG. 10 is a graph depicting the change in current response over time during the application of the input waveform of FIG. 9 in a sample measurement using a primary redox probe and a secondary redox probe (“SRP”). The graph of FIG. 10 is a measurement of the current response of the reaction cell due to the application of a two potential waveform. Therefore, time 0 in FIG. 10 corresponds to time 3.0 in FIG. 9. The following description provides several exemplary methods of using the SRP data to determine a hematocrit correction factor. Numerous methods, however, can be used to process the data resulting from the input waveform of FIG. 9 and the following examples are intended to be non-limiting.

In one embodiment, the current response signal, such as the one depicted in FIG. 10, is measured at a specific point during the time of the second pulse. The magnitude of that measurement is subtracted from the magnitude of the current response signal measured at a specific point during the first pulse. This process can be described by the following equation parameters. Two voltage potential pulses are applied to the reaction cell. A first pulse (of a predetermined voltage magnitude) is applied for the time interval from time zero to time X. A second pulse (of a second predetermined voltage magnitude) is then applied for the time interval described by the range of time X to time X+Y.

Two current response measurements are recorded at two times, time t₁ and time t₂ where 0≦t₁≦X and X≦t₂≦X+Y. The two current response values are described as I(t₁) and I(t₂). Therefore, in the numerical process described above, a hematocrit correction factor is obtained by subtracting I (t₂) from I (t₁), giving a value V. Value V is then compared with a known standard to determine the particular hematocrit correction factor.

In an additional method, the magnitude of the current response from the second pulse is recorded at two points and the slope between those two points is determined. This slope is divided by the magnitude of the current response value measured at the end of the first pulse. This value obtained is then compared with a known standard to determine the particular hematocrit correction factor. Accordingly, in this method, three current response values are recorded for mathematical analysis.

This approach is detailed in FIG. 10, where the current response is recorded at points A, B, and C depicted therein. The current response value of point A is taken right at the end of the first pulse and the current response values for points B and C are recorded during the second pulse. This process can be described by the following equation parameters. Just as in the previous process, two voltage potential pulses are applied to the reaction cell. A first pulse (of a predetermined voltage magnitude) is applied for the time interval from time zero to time X. A second pulse (of a second predetermined voltage magnitude) is then applied for the time interval described by the range of time X to time X+Y.

Three current response measurements are recorded at three times, time t₁, time t₂, and time t₃ where 0≦t₁≦X and X≦t₂≦t₃≦X+Y. The two current response values are described as I(t₁), I₍ₜ₂₎, and I(t₃) (e.g. the current response values for points A, B, and C respectively). As noted above, the slope between points B and C is calculated and divided by the magnitude of the current response value measured at the end of the first pulse. This new value, value V can be described by the equation:

$$\frac{\vert I ( t 3 ) - I ( t 2 ) \vert / \vert t 3 - t 2 \vert}{\vert I ( t 1 ) \vert}$$

where the numerator defines an absolute value of the slope between points B and C and the denominator defines the absolute value magnitude of the current response value measured at the end of the first pulse.

Therefore, in the numerical process described above, a hematocrit correction factor is obtained by deriving Value V, according the above equation. Since the SRP response is dependent on the blood hematocrit level, a comparison of SRP signal and the primary signal (as provided in each of the methods described) yields a value (i.e. value V) that can be used to correct for any errors due to the blood hematocrit level of the sample. The Value V, is then compared with a known standard to determine the particular hematocrit correction factor for this embodiment.

This measurement may be further refined by taking into account the slope of the signal resulting from the primary measurement. In this example, 4 sample points are taken into consideration for the measurement, T1, T2, T3, and T4. The first is between or equal to 0 and X. The second is also between or equal to 0 and X, but T1<T2. T3 is between or equal to X and Y, as is T4. Again, T3<T4. An equation that can be used to describe this is (T3−(T4−(T1−T2)))/(T3−A*(2*T2−T1)), where A is a constant. Since Time is constant, it is not included in the aforementioned equation, in order to simplify computation. This approach can be shown in FIG. 11, where the first curve represents the primary analyte measurement (e.g. glucose) and the second curve represents the SRP response signal. FIG. 11 is an exemplary current vs. time profile that illustrates the current returned from a gentisic acid SRP test. The first (left-hand) decay is the current generated from the 0.3V pulse, and is the primary measurement. The second (right-hand) decay is the SRP pulse at 0.85V, which is suitable for measuring gentisic acid. In this figure, there are four points marked, which correspond to a particular method for measuring the SRP response. This system is suitable for gentisic acid.

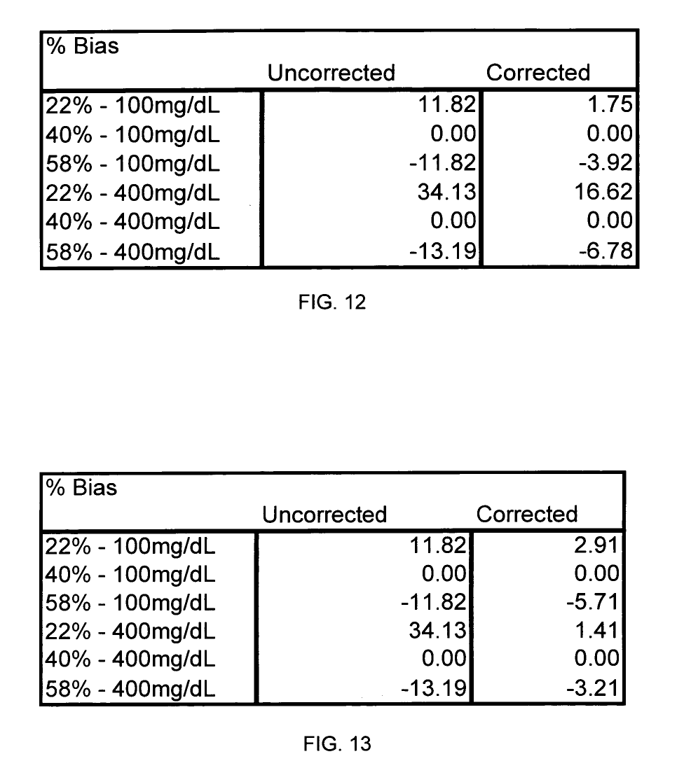

The above measurements may be altered to take into account bias based on glucose level. In biosensors, it can occur that the bias induced by hematocrit can be more severe depending on the concentration of the target analyte. High concentrations of the analyte may have a more severe bias. To correct for this, a function is needed that increases the intensity of the SRP correction effect, but that does not shift the median point. This can be done by altering the second step in the SRP correction process, the comparison of the experimental value to the nominal value. This comparison can be raised to a power that is partially dependent on the current value generated during the primary measurement. This could, for example, take the form (Vₙₒₘᵢₙₐₗ/Vₑₓₚₑᵣᵢₘₑₙₜₐₗ)^{(B+C*T)}, where B and C are numerical constants and T is the value of the current at some time during the primary measurement pulse. Vₙₒₘᵢₙₐₗ is the nominal value of the SRP correction factor. Vₑₓₚₑᵣᵢₘₑₙₜₐₗ is the experimental value obtained for the SRP for a particular sample. The constants can be refined to give good hematocrit correction across a wide range of analyte concentrations. FIGS. 12 and 13 show one set of data that have been treated using gentisic acid as the SRP in two separate methods. The results in FIG. 12 are based on a straight linear correction based on the four-point method outlined in the above paragraphs. The results in FIG. 13 were obtained by adding an exponential correction function to the comparison equation. As can be seen, exponential correction is much more effective in correcting high concentrations, while not sacrificing accuracy at low concentrations. Therefore, it is most preferable to include this in the SRP calculations.

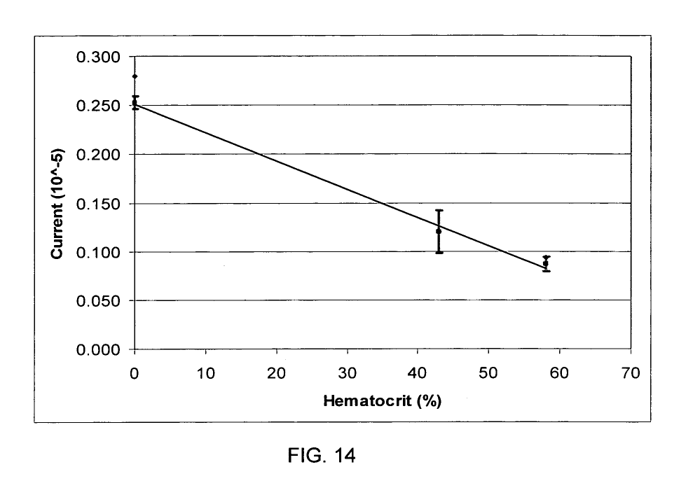

FIG. 14 is a graph depicting the dependence of an SRP mediator, brilliant cresyl blue, on the particular hematocrit level of blood. The graph depicts the current response values for samples with a concentration level of 400 mg/dl. The samples were measured at hematocrit concentration levels of 0, 42, and 58. As seen in FIG. 14, there is a linear relationship between the measured current response and the hematocrit level of the sample. Accordingly, the measured SRP response is clearly dependent on the hematocrit sample level.

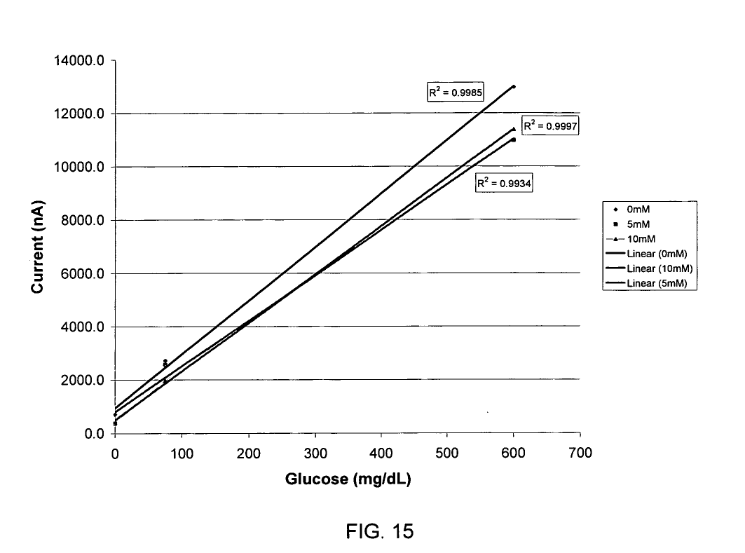

FIG. 15 depicts the relationship between the measured analyte signal magnitude and the actual sample analyte concentration using multiple concentrations of the SRP in the test strip chemistry. In the experiment depicted, strips containing cresyl blue SRP concentrations of 0 mM, 5 mM, and 10 mM added to the standard chemistry formulation were each hand dispensed and tested with samples having 0, 75, and 600 mg/dL glucose concentrations. The results illustrate that the addition of the SRP does not erroneously interfere with the glucose measurement.

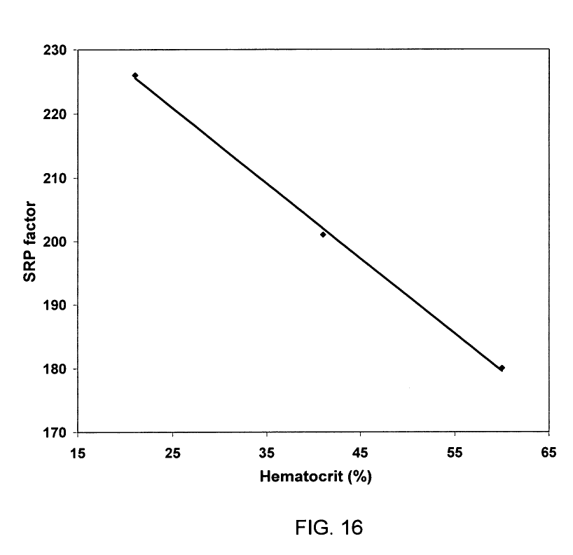

FIG. 16 is a graph showing the derived relationship between a calculated SRP factor and the hematocrit of the sample. In this case, a higher SRP value is indicative of lower hematocrit.

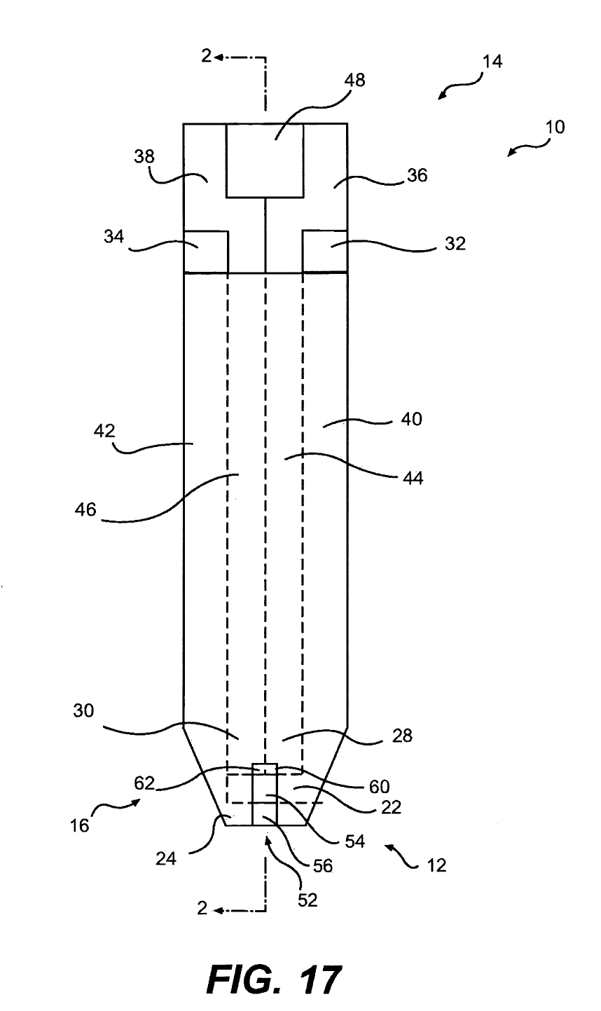

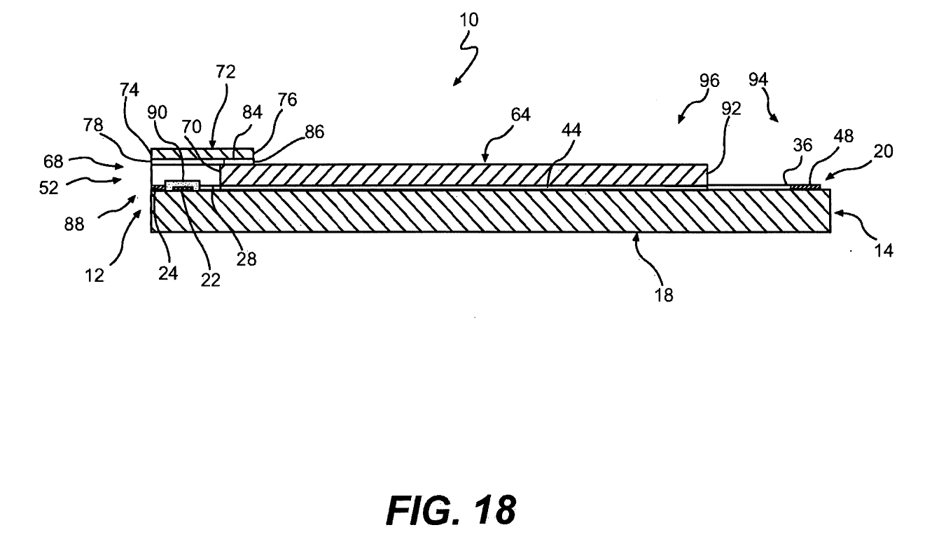

With reference to the drawings, FIGS. 17 and 18 show a test strip 10, in accordance with an exemplary embodiment of the present invention. Test strip 10 preferably takes the form of a generally flat strip that extends from a proximal end 12 to a distal end 14. Preferably, test strip 10 is sized for easy handling. For example, test strip 10 can measure approximately 35 mm long (i.e., from proximal end 12 to distal end 14) and approximately 9 mm wide. However, the strip can be any convenient length and width. For example, a meter with automated test strip handling may utilize a test strip smaller than 9 mm wide. Additionally, proximal end 12 can be narrower than distal end 14 in order to provide facile visual recognition of the distal end. Thus, test strip 10 can include a tapered section 16, in which the full width of test strip 10 tapers down to proximal end 12, making proximal end 12 narrower than distal end 14. As described in more detail below, the user applies the blood sample to an opening in proximal end 12 of test strip 10. Thus, providing tapered section 16 in test strip 10, and making proximal end 12 narrower than distal end 14, assists the user in locating the opening where the blood sample is to be applied. Further, other visual means, such as indicia, notches, contours or the like are possible.

As shown in FIG. 18, test strip 10 can have a generally layered construction. Working upwardly from the bottom layer, test strip 10 can include a base layer 18 extending along the entire length of test strip 10. Base layer 18 can be formed from an electrically insulating material and has a thickness sufficient to provide structural support to test strip 10. For example, base layer 18 can be a polyester material about 0.35 mm thick.

According to the illustrative embodiment, a conductive layer 20 is disposed on base layer 18. Conductive layer 20 includes a plurality of electrodes disposed on base layer 18 near proximal end 12, a plurality of electrical contacts disposed on base layer 18 near distal end 14, and a plurality of conductive regions electrically connecting the electrodes to the electrical contacts. In the illustrative embodiment depicted in FIGS. 17-18, the plurality of electrodes includes a working electrode 22, a counter electrode 24, a fill-detect anode 28, and a fill-detect cathode 30. Correspondingly, the electrical contacts can include a working electrode contact 32, a counter electrode contact 34, a fill-detect anode contact 36, and a fill-detect cathode contact 38. The conductive regions can include a working electrode conductive region 40, electrically connecting working electrode 22 to working electrode contact 32, a counter electrode conductive region 42, electrically connecting counter electrode 24 to counter electrode contact 34, a fill-detect anode conductive region 44 electrically connecting fill-detect anode 28 to fill-detect contact 36, and a fill-detect cathode conductive region 46 electrically connecting fill-detect cathode 30 to fill-detect cathode contact 38. Further, the illustrative embodiment is depicted with conductive layer 20 including an auto-on conductor 48 disposed on base layer 18 near distal end 14.

The next layer in the illustrative test strip 10 is a dielectric spacer layer 64 disposed on conductive layer 20. Dielectric spacer layer 64 is composed of an electrically insulating material, such as polyester. Dielectric spacer layer 64 can be about 0.100 mm thick and cover portions of working electrode 22, counter electrode 24, fill-detect anode 28, fill-detect cathode 30, and conductive regions 40-46, but in the illustrative embodiment does not cover electrical contacts 32-38 or auto-on conductor 48. For example, dielectric spacer layer 64 can cover substantially all of conductive layer 20 thereon, from a line just proximal of contacts 32 and 34 all the way to proximal end 12, except for a slot 52 extending from proximal end 12. In this way, slot 52 can define an exposed portion 54 of working electrode 22, an exposed portion 56 of counter electrode 24, an exposed portion 60 of fill-detect anode 28, and an exposed portion 62 of fill-detect cathode 30.

A cover 72, having a proximal end 74 and a distal end 76, can be attached to dielectric spacer layer 64 via an adhesive layer 78. Cover 72 can be composed of an electrically insulating material, such as polyester, and can have a thickness of about 0.1 mm. Additionally, the cover 72 can be transparent.

Adhesive layer 78 can include a polyacrylic or other adhesive and have a thickness of about 0.013 mm. Adhesive layer 78 can consist of sections disposed on spacer 64 on opposite sides of slot 52. A break 84 in adhesive layer 78 extends from distal end 70 of slot 52 to an opening 86. Cover 72 can be disposed on adhesive layer 78 such that its proximal end 74 is aligned with proximal end 12 and its distal end 76 is aligned with opening 86. In this way, cover 72 covers slot 52 and break 84.

Slot 52, together with base layer 18 and cover 72, defines a sample chamber 88 in test strip 10 for receiving a blood sample for measurement in the illustrative embodiment. Proximal end 12 of slot 52 defines a first opening in sample chamber 88, through which the blood sample is introduced into sample chamber 88. At distal end 70 of slot 52, break 84 defines a second opening in sample chamber 88, for venting sample chamber 88 as sample enters sample chamber 88. Slot 52 is dimensioned such that a blood sample applied to its proximal end 68 is drawn into and held in sample chamber 88 by capillary action, with break 84 venting sample chamber 88 through opening 86, as the blood sample enters. Moreover, slot 52 can advantageously be dimensioned so that the blood sample that enters sample chamber 88 by capillary action is about 1 micro-liter or less. For example, slot 52 can have a length (i.e., from proximal end 12 to distal end 70) of about 0.140 inches, a width of about 0.060 inches, and a height (which can be substantially defined by the thickness of dielectric spacer layer 64) of about 0.005 inches. Other dimensions could be used, however.

A reagent layer 90 is disposed in sample chamber 88. Preferably, reagent layer spreads uniformly throughout the sample cavity. Reagent layer 90 includes chemical constituents to enable the level of glucose in the blood sample to be determined electrochemically. Thus, reagent layer 90 may include an enzyme specific for glucose and a mediator, as described above. In addition, reagent layer 90 may also include other components, such as the secondary redox probe (SRP) materials, buffering materials (e.g., potassium phosphate), polymeric binders (e.g., hydroxypropyl-methyl-cellulose, sodium alginate, microcrystalline cellulose, polyethylene oxide, hydroxyethylcellulose, and/or polyvinyl alcohol), and surfactants (e.g., Triton X-100 or Surfynol 485).

With these chemical constituents, reagent layer 90, including the secondary redox probe material, reacts with glucose in the blood sample in the manner described throughout this application.

As depicted in FIG. 18, the arrangement of the various layers in illustrative test strip 10 can result in test strip 10 having different thicknesses in different sections. In particular, among the layers above base layer 18, much of the thickness of test strip 10 can come from the thickness of spacer 64. Thus, the edge of spacer 64 that is closest to distal end 14 can define a shoulder 92 in test strip 10. Shoulder 92 can define a thin section 94 of test strip 10, extending between shoulder 92 and distal end 14, and a thick section 96, extending between shoulder 92 and proximal end 12. The elements of test strip 10 used to electrically connect it to the meter, namely, electrical contacts 32-38 and auto-on conductor 48, can all be located in thin section 94. Accordingly, the connector in the meter can be sized and configured to receive thin section 94 but not thick section 96, as described in more detail below. This can beneficially cue the user to insert the correct end, i.e., distal end 14 in thin section 94, and can prevent the user from inserting the wrong end, i.e., proximal end 12 in thick section 96, into the meter.

Although FIGS. 17 and 18 illustrate an illustrative embodiment of test strip 10, other configurations, chemical compositions and electrode arrangements could be used.

Different arrangements of fill-detect electrodes 28 and 30 can also be used. In the configuration shown in FIGS. 17 and 18, fill-detect electrodes 28 and 30 are in a side-by-side arrangement. Alternatively, fill-detect electrodes 28 and 30 can be in a sequential arrangement, whereby, as the sample flows through sample chamber 88 toward distal end 70, the sample contacts one of the fill-detect electrodes first (either the anode or the cathode) and then contacts the other fill-detect electrode.

As depicted in the Figures, fill-detect electrodes 28 and 30 are advantageously located on the distal side of reagent layer 90. In this arrangement, the sample introduced into the sample chamber 88 will have traversed reagent layer 90 before reaching fill-detect electrodes 28 and 30. This arrangement beneficially allows the fill-detect electrodes 28 and 30 to indicate not only whether sufficient blood sample is present in sample chamber 88, but also when, concomitantly, the blood sample has sufficiently mixed with the chemical constituents of reagent layer 90. Other configurations are of course possible.

Test Strip Array Configuration

Test strips can be manufactured by forming a plurality of strips in an array along a reel or web of substrate material. The term “reel” or “web” as used herein applies to continuous webs of indeterminate length, or to sheets of determinate length. The individual strips, after being formed, can be separated during later stages of manufacturing. An illustrative embodiment of a batch process of this type is described infra. First, an illustrative test strip array configuration is described.

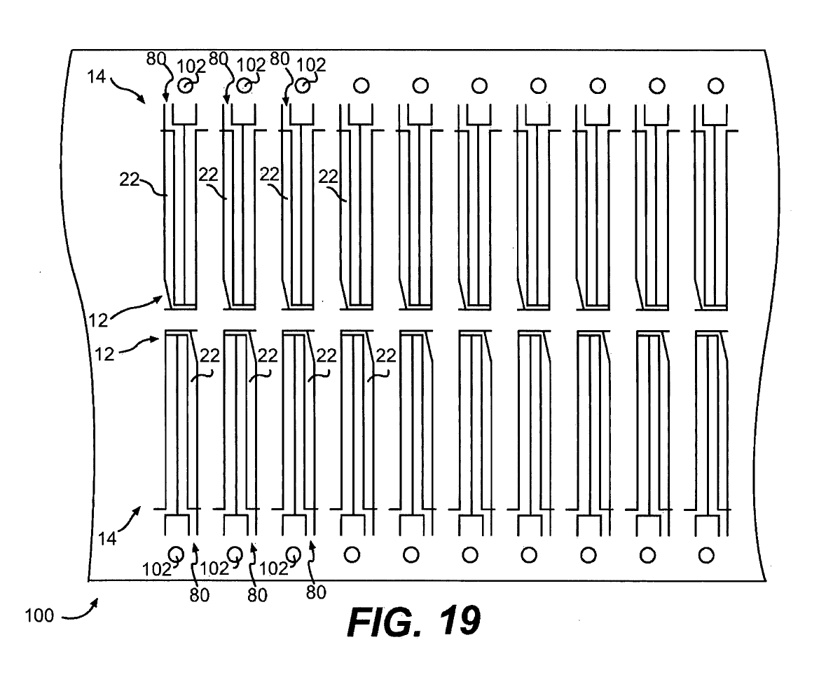

FIG. 19 shows a series of traces 80 formed in a substrate material coated with a conductive layer. Traces 80, formed in the exemplary embodiment by laser ablation, partially form the conductive layers of two rows of ten test strips as shown. In the exemplary embodiment depicted, proximal ends 12 of the two rows of test strips are in juxtaposition in the center of a reel 100. The distal ends 14 of the test strips are arranged at the periphery of reel 100. It is also contemplated that the proximal ends 12 and distal ends 14 of the test strips can be arranged in the center of reel 100. Alternatively, the two distal ends 14 of the test strips can be arranged in the center of reel 100. The lateral spacing of the test strips is designed to allow a single cut to separate two adjacent test strips. The separation of the test strip from reel 100 can electrically isolate one or more conductive components of the separated test strip 10.

As depicted in FIG. 19, trace 80 for an individual test strip forms a plurality of conductive components; e.g., electrodes, conduction regions and electrode contacts. Trace 80 is comprised of individual cuts made by a laser following a specific trajectory, or vector. A vector can be linear or curvilinear, and define spaces between conductive components that are electrically isolating. Generally a vector is a continuous cut made by the laser beam.

The conductive components can be partially or entirely defined by ablated regions, or laser vectors, formed in the conductive layer. The vectors may only partially electrically isolate the conductive component, as the component can remain electrically connected to other components following laser ablation. The electrical isolation of the conductive components can be achieved following “singulation,” when individual test strips are separated from reel or web 100.

FIG. 19 shows a plurality of electrically isolated working electrodes 22. According to the illustrated embodiment, working electrode 22 of an individual test strip can be electrically isolated from the other conductive components during the laser ablation process. It is also contemplated that other conductive components may be electrically isolated during the laser ablation process. For example, fill detect electrodes may be isolated with the addition of one or more vectors.

FIG. 19 also includes registration points 102 at the distal end 14 of each test strip on reel 100. Registration points 102 assist the alignment of the layers during the lamination, punching and other manufacturing processes. It is further contemplated that registration points 102 may be located at locations other than the distal end 14 of each test strip trace 80 on reel 100. High quality manufacturing may require additional registration points 102 to ensure adequate alignment of laminate layers and/or other manufacturing processes, such as, for example, laser ablation of conductive components, reagent deposition, singulation, etc.

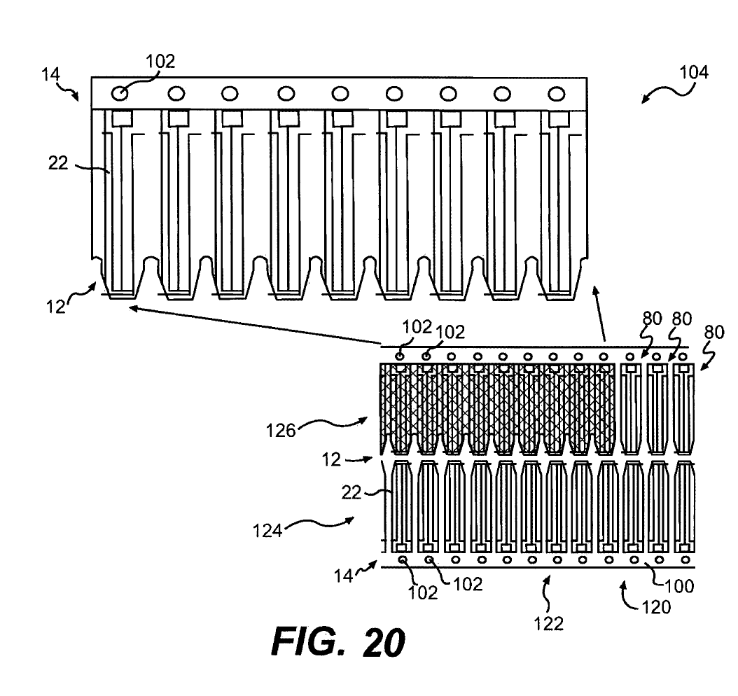

FIG. 20 shows a number of strips forming a card 104 separated from reel 100. Card 104 can contain a plurality of test strips 10 or traces 80, and a plurality of conductive components. In the preferred embodiment card 104 can contain between 6 and 12 test strips 10 or traces 80. In other embodiments, card 104 can contain a plurality of test strips 10 or traces 80. In the illustrated embodiment, card 104 can include a lateral array of test strips 10 or traces 80. In other embodiments, card 104 can include an array or arrays of test strips 10 or traces 80 in longitudinal and/or lateral configurations. It is further contemplated that test strips 10 or traces 80 may be in any arrangement on reel 100 suitable for manufacturing.

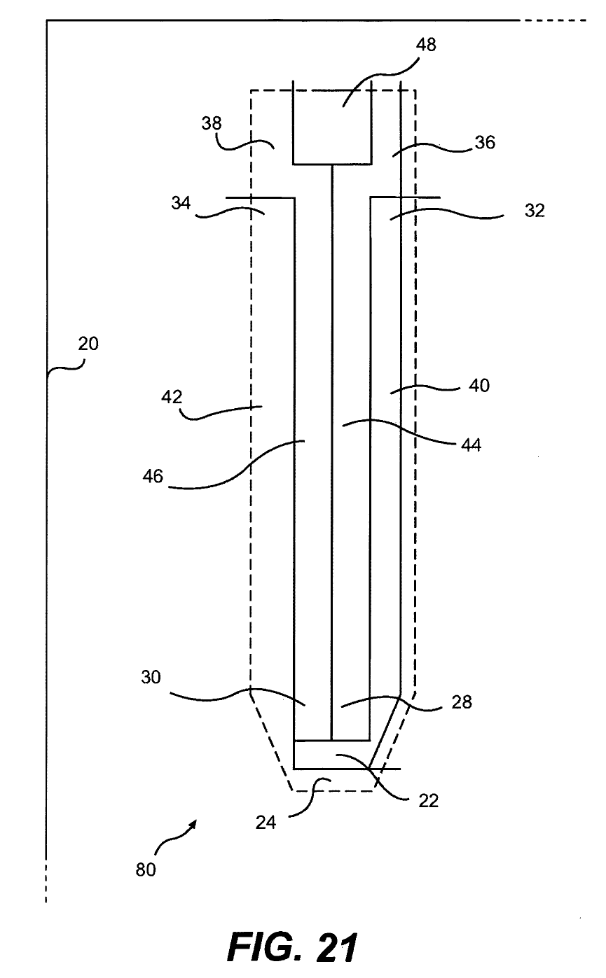

Card 104 contains a plurality of conductive components. Some conductive components can be electrically isolated when the card is removed from the reel. As shown in FIG. 20, working electrode 22 is electrically isolated. Other embodiments could include additional electrically isolated conductive components not shown in FIG. 21. It may be possible to analyze properties of the electrically isolated conductive components to assess the quality of the manufacturing process and strip chemistry application. The efficiency of the quality assessment process can be increased by testing at least one of the plurality of electrically isolated conductive components in order to determine a particular calibration code based on the particular strip chemistry, for example.

Batch Manufacturing of Test Strips

FIGS. 21 through 24 illustrate an exemplary method of manufacturing test strips. Although these figures shows steps for manufacturing test strip 10, as shown in FIGS. 17 and 18, it is to be understood that similar steps can be used to manufacture test strips having other configurations.

With reference to FIG. 20, a plurality of test strips 10 can be produced by forming a structure 120 that includes a plurality of test strip traces 122 on reel 100. Test strip traces 122 include a plurality of traces 80, and can be arranged in an array that includes a plurality of rows. Each row 124 can include a plurality of test strip traces 122.

The separation process can also be used to electrically isolate conductive components of test strip 10. Laser ablation of the conductive layer may not electrically isolate certain conductive components. The non-isolated conductive components may be isolated by the separation process whereby test strips are separated from reel 100. The separation process may sever the electrical connection, isolating the conductive component. Separating test strip 10 can electrically isolate the counting electrode 24, fill detect-anode 28 and fill-detect cathode 30. The separation process can complete the electrical isolation of conductive components by selectively separating conductive components.

Further, the separation process can provide some or all of the shape of the perimeter of the test strips 10. For example, the tapered shape of tapered sections 16 of the test strips 10 can be formed during this punching process. Next, a slitting process can be used to separate the test strip structures 122 in each row 124 into individual test strips 10. The separation process may include stamping, slitting, scoring and breaking, or any suitable method to separate test strip 10 and/or card 104 from reel 100.

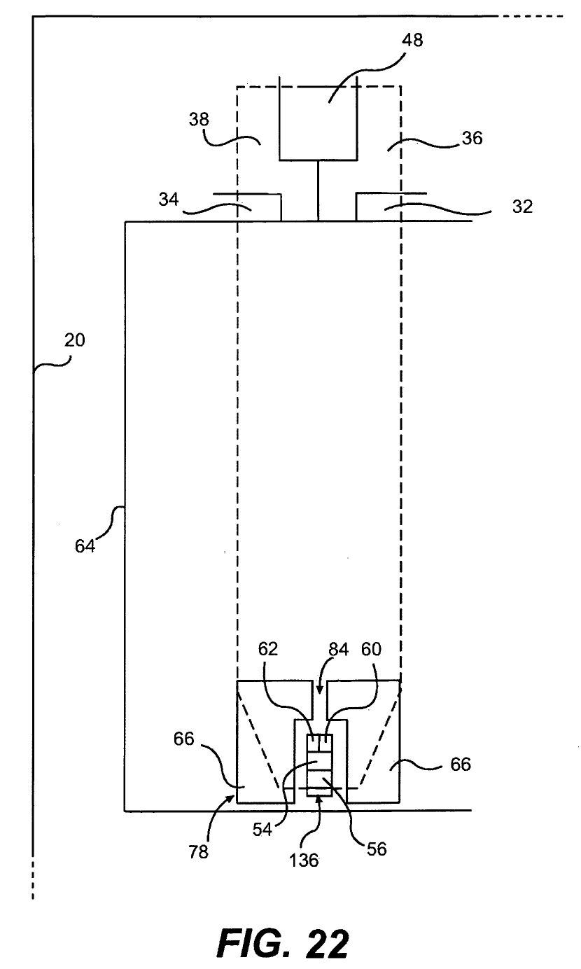

FIGS. 21 and 22 show only one test strip structure (either partially or completely fabricated), in order to illustrate various steps in a preferred method for forming the test strip structures 122. In this exemplary approach, the test strip structures 122 in integrated structure 120 are all formed on a sheet of material that serves as base layer 18 in the finished test strips 10. The other components in the finished test strips 10 are then built up layer-by-layer on top of base layer 18 to form the test strip structures 122. In each of FIGS. 21 and 22, the outer shape of the test strip 10 that would be formed in the overall manufacturing process is shown as a dotted line.

The exemplary manufacturing process employs base layer 18 covered by conductive layer 20. Conductive layer 20 and base layer 18 can be in the form of a reel, ribbon, continuous web, sheet, or other similar structure. Conductive layer 20 can include any suitable conductive or semi-conductor material, such as gold, silver, palladium, carbon, tin oxide and others known in the art. Conductive layer 20 can be formed by sputtering, vapor deposition, screen printing or any suitable manufacturing method. The conductive material can be any suitable thickness and can be bonded to base layer 18 by any suitable means.

As shown in FIG. 21, conductive layer 20 can include working electrode 22, counter electrode 24, fill-detect anode 28, and fill-detect cathode 30. Trace 80 can be formed by laser ablation where laser ablation can include any device suitable for removal of the conductive layer in appropriate time and with appropriate precision and accuracy. Various types of lasers can be used for sensor fabrication, such as, for example, solid-state lasers (e.g. Nd:YAG and titanium sapphire), copper vapor lasers, diode lasers, carbon dioxide lasers and excimer lasers. Such lasers may be capable of generating a variety of wavelengths in the ultraviolet, visible and infrared regions. For example, excimer laser provides wavelength of 248 nm, a fundamental Nd:YAG laser gives 1064 nm, a frequency tripled Nd:YAG wavelength is at 355 nm and a Ti:sapphire laser is at approximately 800 nm. The power output of these lasers may vary and is usually in range 10-100 watts.

The laser ablation process can include a laser system. The laser system can include a laser source. The laser system can further include means to define trace 80, such as, for example, a focused beam, projected mask or other suitable technique. The use of a focused laser beam can include a device capable of rapid and accurate controlled movement to move the focused laser beam relative to conductive layer 20. The use of a mask can involve a laser beam passing through the mask to selectively ablate specific regions of conductive layer 20. A single mask can define test strip trace 80, or multiple masks may be required to form test strip trace 80. To form trace 80, the laser system can move relative to conductive layer 20. Specifically, the laser system, conductive layer 20, or both the laser system and conductive layer 20 may move to allow formation trace 80 by laser ablation. Exemplary devices available for such ablation techniques include Microline Laser system available from LPKF Laser Electronic GmbH (Garbsen, Germany) and laser micro machining systems from Exitech, Ltd (Oxford, United Kingdom).

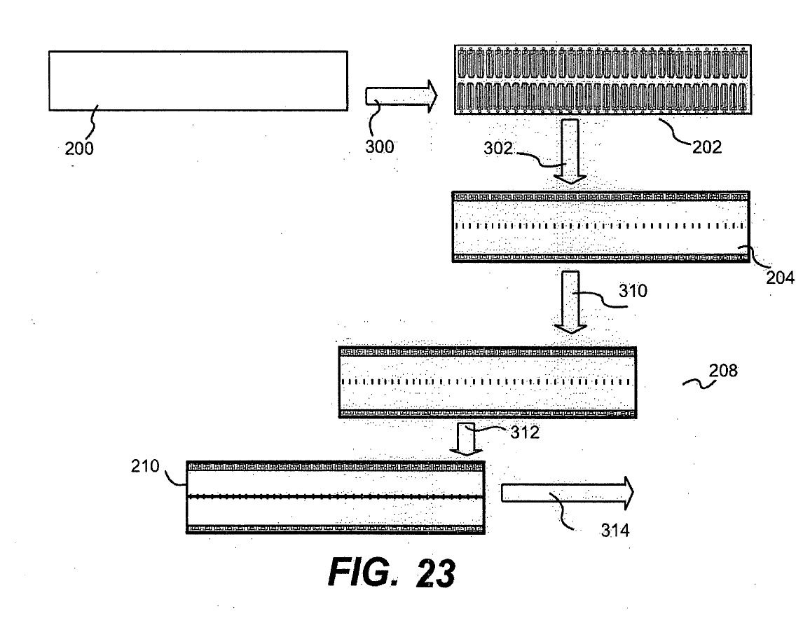

In the next step, dielectric spacer layer 64 can be applied to conductive layer 20, as illustrated in FIG. 22. Spacer 64 can be applied to conductive layer 20 in a number of different ways. In an exemplary approach, spacer 64 is provided as a sheet or web large enough and appropriately shaped to cover multiple test strip traces 80. In this approach, the underside of spacer 64 can be coated with an adhesive to facilitate attachment to conductive layer 20. Portions of the upper surface of spacer 64 can also be coated with an adhesive in order to provide adhesive layer 78 in each of the test strips 10. Various slots can be cut, formed or punched out of spacer 64 to shape it before, during or after the application of spacer layer 64 to conductive layer 20. For example, as shown in FIG. 22, spacer 64 can have a pre-formed slot 136 for each test strip structure. In addition, spacer 64 can include adhesive sections 66, with break 84 there between, for each test strip trace 80. Spacer 64 is then positioned over conductive layer 20, as shown in FIG. 23, and laminated to conductive layer 20. When spacer 64 is appropriately positioned on conductive layer 20, exposed electrode portions 54-62 are accessible through slot 136. Thus, slot 52 in test strip 10 corresponds to that part of slot 136 that remains in test strip 10 after the test strip structures are separated into test strips. Similarly, spacer 64 leaves contacts 32-38 and auto-on conductor 48 exposed after lamination.

Alternatively, spacer 64 could be applied in other ways. For example, spacer 64 can be injection molded onto base layer 18 and dielectric 50. Spacer 64 could also be built up on dielectric layer 50 by screen-printing successive layers of a dielectric material to an appropriate thickness, e.g., about 0.005 inches. A preferred dielectric material comprises a mixture of silicone and acrylic compounds, such as the “Membrane Switch Composition 5018” available from E.I. DuPont de Nemours & Co., Wilmington, Del. Other materials could be used, however.

Reagent layer 90 may then be applied to each test strip structure. In a preferred approach, reagent layer 90 is applied by micropipetting an aqueous composition into sample cavity and drying it to form reagent layer 90. One aqueous composition includes an enzyme specific for glucose, a mediator, and the secondary redox probe (SRP) material. Alternatively, other methods, such as screen-printing, may be used to apply the composition used to form reagent layer 90.

A transparent cover 72 can then be attached to adhesive layer 78. Cover 72 may be large enough to cover multiple test strip structures 122. Attaching cover 72 can complete the formation of the plurality of test strip structures 122. The plurality of test strip structures 122 can then be separated from each other to form a plurality of test strips 10, as described above.

Quality Control Testing of Test Strips

FIG. 23 shows a further illustrative embodiment of a test strip manufacturing method. The manufacturing method utilizes a web 200 containing conductive layer 20 and base layer 18. Conductive layer 20 and base layer 18 can be any suitable material. Web 200 can be any dimension suitable for production of the test strips. Web 200 is passed through any suitable device and ablated by process 300.

Ablation 300 can include any suitable ablation process capable of forming conductive components in conductive layer 20. In the illustrative embodiment, ablation 300 is achieved by laser ablation. The ablation process may not electrically isolate all conductive components. For example, counter electrode 24 may not be isolated by laser ablation but can be isolated by subsequent separation from web 200. In the illustrative embodiment, working electrode 22 is electrically isolated during ablation process 300. The counter electrode 24, fill-detect anode 28 and fill-detect cathode 30 may not be electrically isolated during ablation process 300. Specifically, subsequent separation process can electrically isolate the counter electrode 24, fill-detect anode 28 and fill-detect cathode 30.

Web 200 can be passed through any suitable ablation device at speeds sufficient to produce an appropriate rate of test strip production. The ablation process can be sufficiently rapid to allow the continuous movement of web 200 through the laser ablation device. Alternatively, web 200 can be passed through the ablation device in a non-continuous (i.e., start-and-stop) manner.

The properties of the conductive components formed by ablation process 300 can be analyzed during or following ablation process 300. Analysis of ablation process 300 can include optical, chemical, electrical or any other suitable analysis means. The analysis can monitor the entire ablation process, or part of the ablation process. For example, the analysis can include monitoring vector formation to ensure the dimensions of the formed vector are within predetermined tolerance ranges.

Quality control analysis, which can be performed during or upon completion of the manufacturing process, can also include monitoring the effectiveness and/or efficiency of the vector formation process. In particular, the width of the resulting vectors can be monitored to ensure acceptable accuracy and precision of the cuts in conductive layer 20. For example, the quality of the laser ablation process can be analyzed by monitoring the surface of conductive layer 20 and/or base layer 18 following ablation. Partial ablation of base layer 18 can indicate that the laser power is set too high or the beam is traveling too slowly. By contrast, a partially ablated conductive layer may indicate insufficient laser power or that the beam is traveling too quickly. Incomplete ablation of gaps may result in the formation of vectors that are not electrically isolating between conductive components.

In the illustrative embodiment, the dimensions of working electrode 22 can be analyzed to determine the quality of the manufacturing process. For example optical analysis (not shown) can monitor the width of working electrode 22 to ensure sufficient accuracy of ablation process 300. Further, the alignment of working electrode 22 relative to registration points 102 can be monitored. Optical analysis can be performed by using VisionPro system from Cognex Vision Systems (Natick, Mass.).

As described above, the ablation process produces an array of test strips 202 on web 200. Following formation of test strip array 202 and corresponding conductive components, dielectric spacer 64 is laminated to conductive layer 20. The spacer lamination process 302 can include registration points 102 to correctly align spacer layer 64 with conductive layer 20. Spacer 64 may contain registration points 102 corresponding to registration points 102 of test strip array 202. The correct alignment of the layers will position slot 136 over the electrodes as indicated in FIG. 22, forming a three-layer laminate 204.

Following the formation of three-layer laminate 204, the chemistry can be applied to three-layer laminate 204 by a chemistry application process 310. The resulting laminate 208 can contain any appropriate reagent suitable for the specific test strip. The reagent application process 310 can include any appropriate process, such as, for example, the application of an SRP component.

Following reagent application 310, cover 72 can be applied to laminate 208 using any appropriate cover application process 312. Cover 72 may be centered on laminate 208. The resulting laminate 210 can be tested to ensure the quality of the cover application process 312. For example, optical means can be used to monitor the alignment of the cover to laminate 208. Alternatively, laminate 210 can be tested to ensure the quality of any upstream manufacturing process as described previously. Following cover application 312, laminate 210 can be subject to quality control testing as mentioned above. For example, quality control analysis can monitor the effectiveness of the chemistry application. Specifically, optical analysis may be required to determine the extent of reagent covering working electrode 22 and/or counter electrode 24. Alternatively, any previous or upstream manufacturing process can be tested following formation of laminate 210. In addition, following the formation of laminate 210 an entire strip lot can be analyzed to determine a particular lot code to be associated with that particular strip lot. For example, during process 314, the resulting laminate 210 (or even a portion, such as card 104, depicted in FIG. 20) could be analyzed to determine a lot code that includes information regarding a particular calibration code used by a meter to produce accurate sample measurements. The coded information may be any suitable identifier containing batch, lot, manufacturing, and/or other information pertinent to the manufacturing process, test strip 10, and/or the underlying meter.

The resulting coded assembled web containing test strips 10 with coded numbers, for example, can be passed into a device to form singulated test strips. The singulation process, for example, can include singulation of the individual test strips and/or any appropriate handling or packaging process. The singulated test strips (not shown) can be further processed if required. For example, test strips 10 of the coded assembled web can be singulated and placed in storage vials.

### CONCLUSION

In summary, the SRP correction approach has a number of advantages. It can be applied to many biosensors, not just oxidation-based glucose sensors. It has a high degree of accuracy and precision in regards to measuring and correcting for hematocrit, and can even improve % CVs within a sample set, enhancing not only hematocrit bias correction, but the actual overall precision of the measurement device. The amount of test time added due to the SRP is negligible and is often not noticeable to the end user.

Further, this method negates the need for a complicated table or multivariate matrix of primary analyte signal versus SRP signal, as even a simple correction algorithm will produce an output which does not vary with respect to analyte signal across a wide range of analyte concentration, but which does vary consistently and accurately with hematocrit.

While various substances are described as possible candidates for use as an SRP, they are not intended to be limiting of the claimed invention. Unless expressly noted, the particular substances are listed merely as examples and are not intended to be limiting of the invention as claimed. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A biosensor for measuring a constituent concentration in blood, said biosensor comprising:
a sample reception region for receiving a blood sample; and
a reaction reagent system comprising:
an oxidation-reduction enzyme specific for the constituent;
a first electron mediator capable of being reversibly reduced and oxidized such that a first electrochemical signal resulting from the reduction or oxidation is related to the constituent concentration in the blood sample;
a second electron mediator capable of undergoing an electrochemical redox reaction where a second electrochemical signal produced by oxidation or reduction of the second mediator is not directly related to the constituent concentration in the blood sample; and
wherein the second electrochemical signal changes based on the hematocrit level of the blood sample.

2. The biosensor of claim 1, wherein the constituent is glucose.

3. The biosensor of claim 1, wherein the first mediator is a ruthenium containing material.

4. The biosensor of claim 3, wherein the ruthenium containing material comprises hexaamine ruthenium (III) trichloride.

5. The biosensor of claim 1, wherein the second mediator comprises brilliant cresyl blue.

6. The biosensor of claim 1, wherein the second mediator comprises gentisic acid (2,5-dihydroxybenzoic acid).

7. The biosensor of claim 1, wherein the second mediator comprises 2,3,4-trihydroxybenzoic acid.

8. The biosensor of claim 1, wherein the second mediator does not interfere with the first electrochemical signal.

9. The biosensor of claim 1, wherein the second mediator is oxidized or reduced in a potential range distinguishable from that of the first mediator.

10. The biosensor of claim 1, wherein the second electron mediator is oxidized or reduced at a potential having a magnitude at least 0.2 volts greater or less than that used to oxidize or reduce the first electron mediator.

11. The biosensor of claim 1, wherein the first and second electrochemical signals are electric current signals obtained through multi-step chronoamperometry.

12. The biosensor of claim 1, wherein the first and second electrochemical signals are electric current signals obtained through square wave voltammetry.

13. The biosensor of claim 1, wherein the first and second electrochemical signals are electric current signals obtained through differential pulse amperometry.

14. The biosensor of claim 1, wherein the first and second electrochemical signals are electric current signals obtained through cyclic voltammetry.

15. A method for determining a constituent concentration in blood, the method comprising:
(a) introducing the blood sample into an electrochemical cell comprising:
(i) spaced apart working and counter electrodes; and
(ii) a redox reagent system comprising an enzyme;
a first electron mediator capable of being reversibly reduced and oxidized such that a first electrochemical signal resulting from the reduction or oxidation is related to the constituent concentration in the blood sample; and
a second electron mediator capable of capable of undergoing an electrochemical redox reaction where a second electrochemical signal produced by oxidation or reduction of the second mediator is not directly related to the constituent concentration in the blood sample and changes based on the hematocrit level of the blood sample;
(b) obtaining the first electrochemical signal;
(c) obtaining the second electrochemical signal;
(d) determining an initial value corresponding to the constituent concentration of the sample using data from the first electrochemical signal; and
(e) correcting the initial value corresponding to the constituent concentration of the sample to remove an effect of the hematocrit level of the sample using a statistical correlation algorithm and data from the second electrochemical signal.

16. The method of claim 15, wherein the constituent is glucose.

17. The method of claim 15, wherein correcting the initial value comprises:
deriving a preliminary constituent concentration from the first and second signals; and
multiplying the preliminary constituent concentration by a correction factor based on the second electrochemical signal to derive the constituent concentration in the sample, corrected to offset an effect of the hematocrit level of the blood sample.

18. The method of claim 15, wherein the statistical correlation comprises determining a slope of the second electrochemical signal.

19. The method of claim 15, wherein the statistical correlation comprises determining a slope of both the first and second electrochemical signals.

20. The method of claim 15, wherein the first electrochemical signal is obtained by applying to the electrochemical cell, a first electric potential of a magnitude capable of oxidizing or reducing the first electron mediator and not capable of oxidizing or reducing the second electron mediator.

21. The method of claim 20, wherein the second electrochemical signal is obtained by applying to the electrochemical cell, a second electric potential of a magnitude capable of oxidizing or reducing the second electron mediator and not capable of oxidizing or reducing the first electron mediator.

22. The method of claim 15, wherein the second electron mediator is oxidized or reduced at a potential having a magnitude at least 0.2 volts greater or less than that used to oxidize or reduce the first electron mediator.

23. The method of claim 15, wherein obtaining the first and second electrochemical signals comprises using multi-step chronoamperometry.

24. The method of claim 15, wherein obtaining the first and second electrochemical signals comprises using square wave voltammetry.

25. The method of claim 15, wherein obtaining the first and second electrochemical signals comprises using differential pulse amperometry.

26. The method of claim 15, wherein obtaining the first and second electrochemical signals comprises using cyclic voltammetry.

27. The method of claim 15, wherein the second electron mediator comprises brilliant cresyl blue.

28. The method of claim 15, wherein the second electron mediator comprises gentisic acid (2,5-dihydroxybenzoic acid).

29. The method of claim 15, wherein the second electron mediator comprises 2,3,4-trihydroxybenzoic acid.

30. A method for determining the hematocrit corrected concentration of an analyte in a physiological sample, said method comprising:
(a) introducing the physiological sample into an electrochemical cell comprising:
(i) spaced apart working and counter electrodes; and
(ii) a redox reagent system comprising an enzyme and a mediator;
(b) applying a first electric potential to the reaction cell and measuring cell current during a first 50 milliseconds of the first electric potential as a function of time to obtain a first time-current transient;
(c) applying a second electric potential to said cell, and measuring cell current as a function of time to obtain a second time-current transient;
(d) deriving a preliminary analyte concentration from said first and second time-current transients; and
(e) multiplying the preliminary analyte concentration by a hematocrit correction factor based on the first and second time-current transient to derive the hematocrit corrected analyte concentration in said sample; whereby the hematocrit corrected concentration of said analyte in said sample is determined.

31. The method of claim 30, wherein the first electric potential is a negative electric pulse and the second electrical potential is a positive electrical pulse.

32. The method of claim 30, wherein the first electric potential is an applied pulse having a duration of about 1-10 milliseconds.

33. The method of claim 30, wherein the preliminary analyte concentration is determined in part based on a current time transient value as sampled at an end of the applied pulse of the first electric potential.

34. The method of claim 30, wherein the second electric potential is an applied pulse or about 1-4 seconds.

35. The method of claim 30, wherein the preliminary analyte concentration is determined in part based on a current time transient value as sampled at an end of the applied pulse of the second electric potential.

36. A method of manufacturing a plurality of test strips, comprising:
forming a web containing a conductive layer and a base layer;
partially forming said plurality of test strips by electrically isolating a first group of conductive components in the conductive layer using a first process;
subsequently forming said plurality of test strips by electrically isolating a second group of conductive components in the conductive layer using a second process wherein first and second processes are not the same; and
forming a reagent layer including:
an enzyme;
a first electron mediator capable of being reversibly reduced and oxidized such that a first electrochemical signal resulting from the reduction or oxidation is related to the constituent concentration in the blood sample; and
a second electron mediator capable of undergoing an electrochemical redox reaction where a second electrochemical signal produced by oxidation or reduction of the second mediator is not directly related to the constituent concentration in the blood sample and changes based on the hematocrit level of the blood sample.

37. The method of claim 36, wherein the web includes a plurality of registration points.

38. The method of claim 36, wherein the first process includes a laser ablation process.

39. The method of claim 36, wherein the second process includes a separation process.

40. The method of claim 39, wherein the separation process includes stamping.

41. The method of claim 39, wherein the separation process includes separating a plurality of test strips from the web.

42. The method of claim 37, wherein the plurality of registration points are separated by approximately 9 mm.

43. The method of claim 37, wherein the plurality of registration points are separated by less than approximately 9 mm.

44. The method of claim 36, wherein the first group of conductive components are separated by less than approximately 9 mm.
